(19) United States
(12) Patent Application Publication
(10) Pub. No.: US 2025/0339371 A1
(43) Pub. Date: Nov. 6, 2025

(54) LIQUID FORMULATION OF IL-22R ANTIBODY

(71) Applicant: LEO PHARMA A/S, Ballerup (DK)

(72) Inventors: Heidi Westh BAGGER, Ballerup (DK); Stefano COLOMBO, Ballerup (DK)

(21) Appl. No.: 18/871,181

(22) PCT Filed: May 30, 2023
(86) PCT No.: PCT/EP2023/064405

§ 371 Date: Dec. 3, 2024

(30) Foreign Application Priority Data
Jun. 3, 2022 (EP) 22177269.2

(51) Int. Cl. A61K 9/08; A61K 9/00; A61K 47/10; A61K 47/18; A61K 47/20; A61K 47/22; A61K 47/26; C07K 16/28

(52) U.S. Cl. CPC A61K 9/08; A61K 9/0019; A61K 47/10; A61K 47/183; A61K 47/20; A61K 47/22; A61K 47/26; C07K 16/2866

(57) ABSTRACT

The disclosure relates to aqueous liquid antibody formulations comprising an antibody that are in terms of stability, osmolality, viscosity and syringe ability is suitable for injection.

### BACKGROUND OF THE INVENTION

The present disclosure relates to aqueous liquid antibody formulations and other protein formulations that are in terms of stability, osmolality, viscosity and syringe ability, suitable for injection. Antibody and other proteins may be administrated to patients via subcutaneous injection. To ensure patient convenience, it is desirable that subcutaneous injection dosage forms are not associated with injection pain or injection difficulties, hence the formulation should preferably be isotonic, be dosed with relatively small injection volumes while having a concentration of the active component which is sufficiently high to achieve desirable clinical dose and desirable clinical results. Increased protein concentration is associated with exponential increase in viscosity which results in increased manufacturing risks, increased risks associated with identifying optimal device and needle solutions, reduced injectability through thin needles, and hence potential reduced convenience for patients during injection. Furthermore, formulations must be stable as such and provide a sufficient stabilizing environment for the protein/antibody in order to avoid structural degradation and protein aggregation to maintain the desired clinical effect of the product after storage, providing an acceptable shelf life of the product.

Increased storage stability of proteins can be achieved by lyophilization/freeze drying, where water is removed (sublimated) and the formulation is changed from an aqueous formulation of the protein into solid, and in principle water free matrix consisting of protein and excipients. However, such lyophilized products require a reconstitution step prior to injection, and are not suitable for prefilled syringes or autoinjectors. Therefore, liquid formulations are preferred for patient convenience.

The present invention provides liquid, thermostable, formulation of an IL-22R antibody useful in the treatment of dermatological conditions, such as atopic dermatitis. The invention also discloses a stable high concentration formulation allowing for small injection volumes or higher doses.

The use of stable liquid formulations is advantageous in the clinical setting and for patient compliance. In contrast to for example lyophilized products which need to be reconstituted before use. The stable liquid formulations can be used in prefilled syringes or autoinjectors. High concentration formulations of antibodies may be desirable in order to reduce the injection volume, in particular for products intended for subcutaneous dosing. However, high concentration antibody formulations are often challenged by insufficient stability e.g. due to protein aggregation and by viscosity exceeding thresholds for simple manufacturing and injection.

The present invention presents formulations solving the above challenges.

### SUMMARY OF THE INVENTION

The invention provides, the following embodiments, all of them to be understood as independent embodiments or as embodiments dependent on any of the other embodiments listed:

A liquid pharmaceutical formulation comprising an IL-22R antibody at a concentration 150±15 mg/mL-225 mg/ml±25 mg/mL, and further comprising:
one or more disaccharides,
one or more amino acids,
optionally a surfactant,
at pH 5.5-6.5, having an osmolality of 280-450 mOsm/kg.

A liquid pharmaceutical formulation, comprising an IL-22R antibody at a concentration 150±15 mg/mL-200 mg/ml±25 mg/mL, and further comprising:
one or more disaccharides,
one or more amino acids,
optionally a surfactant,
at pH 5.5-6.5, having an osmolality of 280-450 mOsm/kg.

A liquid pharmaceutical formulation, comprising an IL-22R antibody at a concentration 150±15 mg/mL-200 mg/ml±25 mg/mL, and further comprising:
one or more disaccharides,
one or more amino acids,
a buffer,
an antioxidant,
optionally a viscosity lowering agent,
optionally a surfactant,
at pH 5.5-6.5, having an osmolality of 280-450 mOsm/kg.

A liquid pharmaceutical formulation, comprising an IL-22R antibody at a concentration 150±15 mg/mL-200 mg/mL±25 mg/mL, and further comprising:
one or more disaccharides at total concentrations of 60-260 mM,
one or more amino acids at total concentration of 40-140 mM,
optionally a surfactant,
at pH 5.5-6.5, having an osmolality of 280-450 mOsm/kg.

A liquid pharmaceutical formulation, comprising an IL-22R antibody at a concentration 150±15 mg/mL-200 mg/ml±25 mg/mL, and further comprising:
one or more disaccharides at total concentrations of 60-260 mM,
one or more amino acids at total concentration of 40-140 mM,
an antioxidant,
a buffer,
optionally a viscosity lowering agent,
optionally a surfactant,
at pH 5.5-6.5, having an osmolality of 280-450 mOsm/kg.

A liquid pharmaceutical formulation, comprising an IL-22R antibody at a concentration 150±15 mg/mL-200 mg/mL±25 mg/mL, and further comprising:
one or more disaccharides at total concentrations of 60-260 mM,
one or more amino acids, selected from the group consisting of glycine, proline, lysine, glutamic acid, methionine, arginine, aspartic acid, and histidine.
at total concentration of 40-140 mM,
optionally a surfactant,
at pH 5.5-6.5, having an osmolality of 280-450 mOsm/kg.

A liquid pharmaceutical formulation, comprising an IL-22R antibody at a concentration 150±15 mg/mL-200 mg/mL±25 mg/mL, and further comprising:
one or more disaccharides at total concentrations of 60-260 mM,
one or more amino acids selected from the group consisting of glycine, proline, lysine, glutamic
acid, methionine, arginine, aspartic acid, and histidine.
at total concentration of 40-140 mM, wherein the amino acids functions as stabiliser, anti-oxidant,
viscosity lowering agent and buffer,
optionally a surfactant,
at pH 5.5-6.5, having an osmolality of 280-450 mOsm/kg.

A stable liquid pharmaceutical formulation, comprising an IL-22R antibody at a concentration 150±15 mg/mL-200 mg/ml±25 mg/mL, and further comprising:
one or more disaccharides at total concentrations of 60-260 mM,
one or more amino acids selected from the group consisting of glycine, proline, lysine, glutamic acid, methionine, arginine, aspartic acid, and histidine, at total concentration of 40-140 mM,
wherein
proline and/or glycine are stabilisers,
methionine is an anti-oxidant,
arginine is a viscosity lowering agent,
histidine is a buffer, and
optionally a surfactant,
at pH 5.5-6.5, having an osmolality of 280-450 mOsm/kg.

A stable liquid pharmaceutical formulation, comprising an IL-22R antibody at a concentration 150±15 mg/mL-200 mg/mL±25 mg/mL, and further comprising:
one or more disaccharides at total concentrations of 60-260 mM,
one or more amino acids selected from the group consisting of glycine, proline, lysine, glutamic acid, methionine, arginine, aspartic acid, and histidine.
at total concentration of 40-140 mM, wherein
proline and/or glycine are stabilisers,
methionine is an anti-oxidant,
arginine is a viscosity lowering agent,
histidine is a buffer, and
optionally a surfactant,
at pH 5.5-6.5, having an osmolality of 280-450 mOsm/kg.

A stable liquid pharmaceutical formulation, comprising an IL-22R antibody at a concentration 150±15 mg/mL-200 mg/mL±25 mg/mL, and further comprising:
one or more disaccharides at total concentrations of 60-260 mM,
proline and/or glycine are present at a concentration of 0-80 mM,
methionine is present in a concentration of 5-30 mM,
arginine is present in a concentration of 0-100 mM,
histidine is present in a concentration of 0-30 mM, and
optionally a surfactant,
at pH 5.5-6.5, having an osmolality of 280-450 mOsm/kg.

A stable liquid pharmaceutical formulation, comprising an IL-22R antibody at a concentration 150±15 mg/mL-225 mg/mL±25 mg/mL, and further comprising:
one or more disaccharides at total concentrations of 60-260 mM,
glycine is present at a concentration of 0-80 mM,
methionine is present in a concentration of 5-30 mM,
arginine is present in a concentration of 0-100 mM,
histidine is present in a concentration of 0-30 mM, and
optionally a surfactant,
at pH 5.5-6.5, having an osmolality of 280-450 mOsm/kg.

A stable liquid pharmaceutical formulation, comprising an IL-22R antibody at a concentration 150±15 mg/mL-200 mg/ml±25 mg/mL, and further comprising:
one or more disaccharides at total concentrations of 60-260 mM,
glycine is present at a concentration of 0-80 mM,
methionine is present in a concentration of 5-30 mM,
arginine is present in a concentration of 0-100 mM,
histidine is present in a concentration of 0-30 mM, and
optionally a surfactant,
at pH 5.5-6.5, having an osmolality of 280-450 mOsm/kg.

The liquid pharmaceutical formulation according to the embodiment above wherein the viscosity is below 25 cP at 20-25° C.

The liquid pharmaceutical formulation according to the embodiment above wherein the viscosity is below 20 cP at 20-25° C.

The liquid pharmaceutical formulation according to any of the embodiments above which is stable at 5° C. for at least 3 years in maintaining the high molecular weight products below 5%.

The liquid pharmaceutical formulation according to any of the embodiments above which is stable at 5° C. for at least 2 years in maintaining the high molecular weight products below 5%.

The liquid pharmaceutical formulation according to any of the embodiments above wherein the formulation contains a histidine buffer.

The liquid pharmaceutical formulation according to the embodiment above wherein histidine is present in a concentration of about 10-30 mM

The liquid pharmaceutical formulation according to the embodiment above wherein histidine is present in a concentration of about 20 mM

The liquid pharmaceutical formulation according to any of the embodiments above, wherein the disaccharide is present in a concentration of about 80-240 mM

The liquid pharmaceutical formulation according to any of the embodiments above, wherein the disaccharide is present in a concentration of about 100-220 mM

The liquid pharmaceutical formulation according to any of the embodiments above, wherein the disaccharide is present in a concentration of about 120-200 mM

The liquid pharmaceutical formulation according to any of the embodiments above, wherein the disaccharide is present in a concentration of about 140-180 mM

The liquid pharmaceutical formulation according to any of the embodiments above, wherein the disaccharide is present in a concentration of about 60-120 mM

The liquid pharmaceutical formulation according to any of the embodiments above, wherein the disaccharide is present in a concentration of about 80-110 mM

The liquid pharmaceutical formulation according to any of the embodiments above, wherein the disaccharide is present in a concentration of about 100-180 mM

The liquid pharmaceutical formulation according to any of the embodiments above, wherein the disaccharide is trehalose or sucrose.

The liquid pharmaceutical formulation according to the embodiment above wherein the disaccharide is trehalose.

The liquid pharmaceutical formulation according to any of the embodiments above, wherein the amino acid is selected from the group glycine, proline, lysine, glutamic acid, methionine, arginine, aspartic acid, and histidine.

The liquid pharmaceutical formulation according to the embodiment above wherein the amino acid is glycine, methionine, arginine and histidine.

The liquid pharmaceutical formulation according to any of the embodiments above, wherein the viscosity lowering agent is arginine.

The liquid pharmaceutical formulation according to any of the embodiments above, wherein the antioxidant is methionine.

The liquid pharmaceutical formulation according any of the embodiments above wherein the anti-oxidant methionine is present in a concentration of 10-30 mM.

The liquid pharmaceutical formulation according any of the embodiments above wherein the anti-oxidant methionine is present in a concentration of 20 mM.

The liquid pharmaceutical formulation according to the embodiment above, wherein the surfactant is present in a concentration of 0.01-0.08% (w/w), 0.01-0.06% w/w, 0.01-0.04% (w/w), 0.01-0.03%, 0.01-0.02% (w/w) or 0.02% (w/w).

The liquid pharmaceutical formulation according to any of the embodiments above, wherein the surfactant is polysorbate 20, polysorbate 80 or poloxamer 188.

The liquid pharmaceutical formulation of any of the embodiments above comprising an IL-22R antibody at a concentration 150 mg/mL±15 mg/mL, and
one or more disaccharides at total concentrations of 180-260 mM,
one or more amino acids at total concentration of 40-120 mM,
an anti-oxidant at a concentration of 5-30 mM,
a viscosity lowering agent at a concentration of 0-100 mM,
optionally a surfactant at a total concentration of 0.01-0.03% (w/w),
a histidine buffer at a concentration of 20 mM,
at pH 5.6-6.5, having a osmolality of 280-450 mOsm/kg

The liquid pharmaceutical formulation of the embodiment above, comprising an IL-22R antibody at a concentration 150 mg/mL±15 mg/mL, and
trehalose at total concentrations of 180-260 mM,
glycine at total concentration of 0-80 mM,
methionine at a concentration of 5-30 mM,
tween 20 at a total concentration of 0.01-0.03% (w/w),
a histidine buffer at a concentration of 20 mM,
at pH 5.6-6.5, having a osmolality of 280-450 mOsm/kg

The liquid pharmaceutical formulation of the embodiment above, comprising an IL-22R antibody at a concentration 150 mg/mL±15 mg/mL, and
sucrose at total concentrations of 180-260 mM,
glycine at total concentration of 0-80 mM,
methionine at a concentration of 5-30 mM,
tween 20 at a total concentration of 0.01-0.04% (w/w),
a histidine buffer at a concentration of 20 mM,
at pH 5.6-6.5, and tonicity suitable for subcutaneous (SC) dosing with an osmolality of 280-450 mOsm/kg

The liquid pharmaceutical formulation of any of the embodiments above, comprising an IL-22R antibody at a concentration 150 mg/ml±15 mg/mL, and
trehalose at total concentrations of about 180 mM,
glycine at total concentration of about 80 mM,
methionine at a concentration of about 20 mM,
tween 20 at a total concentration of about 0.01-0.03% (w/w),
a histidine buffer at a concentration of about 20 mM,
at pH 5.6-6.5.

The liquid pharmaceutical formulation of any of the embodiments above, comprising an IL-22R antibody at a concentration 225 mg/ml±25 mg/mL, and further comprising:
trehalose at total concentrations of 60-100 mM,
methionine at a concentration of 5-30 mM,
viscosity lowering agent at a concentration of 60-100 mM,
glycine at concentration of 0-80 mM
optionally a surfactant
at pH 5.6-6.5, having a tonicity of 280-450 mOsm/kg

The liquid pharmaceutical formulation of any of the embodiments above, comprising an IL-22R antibody at a concentration 200 mg/ml±25 mg/mL, and further comprising:
trehalose at total concentrations of 60-100 mM,
methionine at a concentration of 5-30 mM,
viscosity lowering agent at a concentration of 60-100 mM,
glycine at concentration of 0-80 mM
optionally a surfactant
at pH 5.6-6.5, having a tonicity of 280-450 mOsm/kg

The liquid pharmaceutical formulation according to the embodiment above, comprising an IL-22R antibody at a concentration of 200 mg/ml±25 mg/mL, and further comprising:
trehalose at total concentrations of 80-100 mM,
methionine at a concentration of 5-30 mM,
arginine at a concentration of 50-100 mM,
optionally a surfactant at a total concentration of 0.01-0.03% (w/w),
a histidine buffer at a concentration of 20 mM, at pH 5.5-6.5, having an tonicity of 280-450 mOsm/kg

The liquid pharmaceutical formulation according to any of the embodiments above, comprising an IL-22R antibody at a concentration 200 mg/mL±25 mg/mL, and further comprising:
trehalose at total concentrations of about 100 mM,
methionine at a concentration of about 20 mM,
arginine at a concentration of about 80 mM,
polysorbate 20 at a total concentration of about 0.02% (w/w),
histidine buffer at a concentration of about 20 mM,
at pH 5.5-6.5.

The liquid pharmaceutical formulation according to any of the above embodiments which is stable at 5° C. for about 3 years.

The liquid pharmaceutical formulation according to any of the above embodiments which is stable at 5° C. for about 2 years.

### DETAILED DESCRIPTION OF THE INVENTION

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law

As used herein in the specification and in the claims, the phrase “one or more,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” refers, whether related or unrelated to those elements specifically identified. Thus, as a non-limiting example, “one or more of A and B” (or, equivalently, “one or more of A or B,” or, equivalently “one or more A and/or B”) can refer, in one embodiment, to at least one, optionally including more than one, A, with no B present (and optionally including elements other than B); in another embodiment, to one or more, optionally including at least one, B, with no A present (and optionally including elements other than A); It should also be understood that, unless clearly indicated to the contrary, in any methods claimed herein that include more than one step or act, the order of the steps or acts of the method is not necessarily limited to the order in which the steps or acts of the method are recited.

As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result. The term substantially is therefore used herein to capture the potential lack of completeness inherent in many biological and chemical phenomena

IL-22R (also known as IL-22R1 and IL-22RA) is a type II cytokine receptor selectively expressed on skin and epithelial cells. This receptor mediates signaling via three cytokines: interleukin 22 (IL-22), interleukin 20 (IL-20) and interleukin 24 (IL-24). Cytokine signaling via the IL-22R requires the formation of heterodimeric complexes at the cell surface. IL-22 binds to and signals via a complex consisting of IL-22R and IL-10R (also known as IL-10R2), whereas IL-20 and IL-24 bind to and signal via a heterodimeric complex consisting of IL-22R and IL-20R3 (also known as IL-20R2). The IL-22R antibody described in the present invention, is in clinical development against atopic dermatitis.

The IL-22 receptor antibody is described in WO2018011420 and is described in WO2018011420 by the HC of seq. id. No. 67 and LC of seq. id. No 68, the VH by sequence no. 63 and VL by sequence no 64,

HCDR1 sequence no. 34 (SYDMN), HCDR2 sequence no. 36 (SIYNDASNTAYSDSVKG) and HCDR3 sequence no. 6 (VGFSGTYYSES). LCDR1 sequence no. 16 (QGGYYAH), LCDR2 sequence no. 47 (GQNNRPS) and LCDR3 sequence no. 54 (QSGSSSSNAV). Sequence numbers refers to the numbers of the application above.

Below the sequences no. 67, 68, 64 and 63 are outlined.

| | |
|---|---|
| 67 | 1 QVQLVESGGG LVQPGGSLRL SCAASGFTFS SYDMNWVRQA PGKGLEWVSS |
| | 51 IYNDASNTAY SDSVKGRFTI SRDNSKNTLY LQMNSLRAED TAVYYCAKVG |
| | 101 FSGTYYSESW GQGTLVTVSS ASTKGPSVFP LAPSSKSTSG GTAALGCLVK |
| | 151 DYFPEPVTVS WNSGALTSGV HTFPAVLQSS GLYSLSSVVT VPSSSLGTQT |
| | 201 YICNVNHKPS NTKVDKKVEP KSCDKTHTCP PCPAPELLGG PSVFLFPPKP |
| | 251 KDTLMISRTP EVTCVVVDVS HEDPEVKFNW YVDGVEVHNA KTKPREEQYQ |
| | 301 STYRVVSVLT VLHQDWLNGK EYKCKVSNKA LPAPIEKTIS KAKGQPREPQ |
| | 351 VYTLPPSRDE LTKNQVSLTC LVKGFYPSDI AVEWESNGQP ENNYKTTPPV |
| | 401 LDSDGSFFLY SKLTVDKSRW QQGNVFSCSV MHEALHNHYT QKSLSLSPGK |
| 68 | 1 SYELTQPSSV SVALGQTARI TCQGGYYAHW YQQKPGQAPV LVIYGQNNRP |
| | 51 SGIPERFSGS GAGNTATLTI SRAQAEDEAD YYCQSGSSSS NAVFGGGTKL |
| | 101 TVLGQPKAAP SVTLFPPSSE ELQANKATLV CLISDFYPGA VTVAWKADSS |
| | 151 PVKAGVETTT PSKQSNNKYA ASSYLSLTPE QWKSHRSYSC QVTHEGSTVE |
| | 201 KTVAPTECS |
| 64 | 1 SYELTQPSSV SVALGQTARI TCQGGYYAHW YQQKPGQAPV LVIYGQNNRP |
| | 51 SGIPERFSGS GAGNTATLTI SRAQAEDEAD YYCQSGSSSS NAVFGGGTKL |
| | 101 TVL |
| 63 | 1 QVQLVESGGG LVQPGGSLRL SCAASGFTFS SYDMNWVRQA PGKGLEWVSS |
| | 51 IYNDASNTAY SDSVKGRFTI SRDNSKNTLY LQMNSLRAED TAVYYCAKVG |
| | 101 FSGTYYSESW GQGTLVTVSS |

According to the present invention the IL-22 receptor antibody as tested in the examples below is defined by:

SEQ ID No 1:
SYDM
SEQ ID No 2:
SIYNDASNTAYSDSVKG
SEQ ID No 3:
VGFSGTYYSES
SEQ ID No 4:
QGGYYAH
SEQ ID No 5:
GQNNRPS
SEQ ID No 6:
QSGSSSSNAV
Heavy Chain (HC)
SEQ ID No 7:
QVQLVESGGG LVQPGGSLRL SCAASGFTFS SYDMNWVRQA
PGKGLEWVSS IYNDASNTAY SDSVKGRFTI SRDNSKNTLY
LQMNSLRAED TAVYYCAKVG FSGTYYSESW GQGTLVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYQ STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK
Light chain (LC)
SEQ ID No 8:
SYELTQPSSV SVALGQTARI TCQGGYYAHW YQQKPGQAPV
LVIYGQNNRP SGIPERFSGS GAGNTATLTI SRAQAEDEAD
YYCQSGSSSS NAVFGGGTKL TVLGQPKAAP SVTLFPPSSE
ELQANKATLV CLISDFYPGA VTVAWKADSS PVKAGVETTT
PSKQSNNKYA ASSYLSLTPE QWKSHRSYSC QVTHEGSTVE
KTVAPTECS (
Variable heavy chain (VH)
SEQ ID No 9:
QVQLVESGGG LVQPGGSLRL SCAASGFTFS SYDMNWVRQA
PGKGLEWVSS IYNDASNTAY SDSVKGRFTI SRDNSKNTLY
LQMNSLRAED TAVYYCAKVG FSGTYYSESW GQGTLVTVSS
Variable light chain:
SEQ ID No 10:
SYELTQPSSV SVALGQTARI TCQGGYYAHW YQQKPGQAPV
LVIYGQNNRP SGIPERFSGS GAGNTATLTI SRAQAEDEAD
YYCQSGSSSS NAVFGGGTKL TVL

As used herein anti-IL22R, anti IL-22R, anti IL22R anti-IL-22R and the like all refers to aIL-22 receptor antibody, the antibody binding to IL-22 receptor.

A functional variant (equivalent) of IL22R antibody as above, which has essentially the same epitope-binding specificity as anti-IL-22R and exhibits substantially similar bioactivity, is also included in the scope of the present invention and also disclosed in WO2018011420. In some embodiments, a functional variant contains the same regions/residues responsible for antigen-binding, such as the same specificity-determining residues in the CDRs or the whole CDRs. In other embodiments, a functional variant comprises a VH chain that includes a VH CDR1, VH CDR2, and VH CDR3 at least 75% (e.g., 80%, 85%, 15 90%, 95%, or 98%) identical to the corresponding VH CDRs of the antibody, and a VL chain that includes a VL CDR1, VL CDR2, and VL CDR3 at least 75% (e.g., 80%, 85%, 90%, 95%, or 98%) identical to the corresponding VH CDRs as mentioned above. For example, a functional variant may comprise a VH chain that includes up to 5 (e.g., 1, 2, 3, 4, or 5) amino acid residue variations in the VH CDR regions (VH CDR1, CDR2, and/or CDR3 in total) as compared to the VH CDRs, and/or a VL chain that includes up to 5 (e.g., 1, 2, 3, 4, or 5) amino acid residue variations in the VL CDR regions (VL CDR1, CDR2, and/or CDR3 in total) as compared to the VH CDRs as mentioned above. Alternatively, a functional variant comprises a VH chain at least 75% (e.g., 80%, 85%, 90%, 95%, or 98%) identical to the VH chain and a VL chain at least 75% 25 (e.g., 80%, 85%, 90%, 95%, or 98%) identical to the VL chain. The amino acid sequence variations may occur only in one or more of the VH and/or VL framework regions. It is anticipated that antibodies having essentially same characteristics as the above can be useful in the present invention. Some sequence variation while still maintaining the binding characteristics are variations which are covered by the present invention.

Alternatively, or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for 10 altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include 15 substitutions made amongst amino acids within the following groups: (a) M, I, L, V; (b) F, Y, W; (c) K, R, H; (d) A, G; (e) S, T; (f) Q, N; and (g) E, D

The liquid pharmaceutical formulation according to the present invention meet one or more of the following criteria alone or in combination:
Single use liquid formulation for subcutaneous injection
Shelf life stability at least 3 years at 5° C.
In-use stability at room temperature
pH relevant for subcutaneous injection
Osmolality relevant for subcutaneous injection (close to isotonicity upon subcutaneous injection),
Viscosity below 25 cP at 25° C. . . .

The liquid pharmaceutical formulation according to the present invention comprises the IL-22R antibody, a suitable buffer, an antioxidant, one or more suitable stabilizer(s) and a non-ionic surfactant and optionally a viscosity reducing agent. The formulation has a pH of about 5.5-6.5.

In the present invention stability of the formulation means that the antibody has a measurable tendency in the formulation to maintain the monomeric state and/or the physical and chemical structure similar to that of the initial time point or to that of a defined reference point. In the present invention “stable formulation” refers to a formulation where the physical and/or chemical stability parameter of the antibody is about 80-100% of the initially defined value, including retention of stability parameter at least about 99%, 98%, 97%, 96%, 95%, 94%, 93%, 92%, 91%, 90%, 89%, 88%, 87%, 86%, 85%, 84%, 83%, 82%, 81% or 80%. An important stability parameter is related to protein aggregation. The stability of a protein formulation is therefore evaluated by quantifying the percentage of protein aggregates (the percentage of high molecular weight protein, HMWP %), the percentage monomeric protein and the percentage low molecular weight protein (LMWP %) by size exclusion chromatography, as described in the examples section. The thermal stability can be evaluated by storage of samples at elevated temperatures followed by chromatographic analyses. Thermal stability can also be evaluated by scanning fluorimetry where protein denaturation induced by increasing temperature is detected by changes in intrinsic fluorescence, or by light scattering where onset of protein aggregation can be determined by increase in scattering intensity, increase in estimated hydrodynamic radius or estimated molecular weight as described in the example section.

In the context of the present invention and the above “stable” means that the presence of high molecular weight protein is below 5% after 3 years at 5° C., as measured by accelerated conditions such as 6 months at 25° C., HMWP is below 3%, or at even more accelerated conditions such as for 4 weeks at 30° C., HMWP is below 3%.

For formulations without arginine even more accelerated conditions can be used, such as 4 weeks at 40° C., whereafter HMWP is below 4%.

A usual approach for long term stability evaluation is data extrapolation. As example when stored 3 months, 6 months, 1 year and perhaps 2 year shows for example linear correlation between time and measurable stability parameter, extrapolation can be used to predict the amount of degraded and intact monomeric protein at a later time point.

Analysis of the HMWP is performed by size exclusion chromatography. Details are as described in Example 3: Protein samples were analysed by SEC (size exclusion chromatography) using SEC column: Waters BEH 200 SEC, 300 mm×4.6 mm column. Column temperature 25° C. Mobile phase: 100 mM Sodium Phosphate Monobasic Monohydrate and 200 mM Sodium Chloride (NaCl). Flow rate: 0.15 ml/min. Detection 280 nm and 215 nm. SEC integration procedure: HMWP % (total area percent of peaks eluting before the monomer peak), LMWP % (total area percent of peaks eluting after the monomer peak).

In embodiments the formulations are stable for 2-3 years at 5° C.

As used herein, the term “viscosity” refers to the magnitude of internal friction in a fluid and measured values refers to the resistance of a liquid formulation to flow e.g. when injected through a syringe needle during administration to a patient. Viscosity of a protein formulation is affected by the protein concentration and characteristics of the protein itself e.g. the sequence and the effective surface charge, the viscosity of a protein formulation is also affected by other components in the formulation, the ionic strength, pH as well as by the temperature.

A viscosity lowering agent in the context of the present invention is an excipient, which lowers the viscosity of the overall formulation compared to the identical formulation without the viscosity reducing excipient (when measured under the same circumstances, such as same the pH, the same protein concentration, the same temperature and by the same method). A viscosity lowering agent in the context of the present invention can also be an excipient which is substituted by another excipient (to prevent unwanted increased in osmolality) to reduce the viscosity. In embodiment the viscosity lowering agent lowers the viscosity by at least 10%, in embodiments by at least 20%, in embodiments by at least 30%, in embodiments by at least 40%, in embodiments by at least 50%, in embodiments by at least 60% and in other embodiments by at least 70%. “Viscosity” as used herein may be “kinematic viscosity” or “absolute viscosity.”

“Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density:

Absolute Viscosity=Kinematic Viscosity×Density

Absolute viscosity is expressed in units of centipoise (cP). The SI unit of absolute viscosity is Pascal second (Pa*s) or millipascal second (mPa*s), where 1 cP=1 mPa·s.

Viscosity is important for drug substance and drug product production processes for example in relation to ultrafiltration, diafiltration, mixing, and filling into prefilled syringes or autoinjectors. Viscosity is also important for patient compliance in relation to for example needle thickness and pressure applied for injection. Viscosity measurements can be done as described in the examples.

In an aspect of the invention the formulation of the antibody is stable and suitable for administration with for example a prefilled syringe or an autoinjector.

Osmolality is a measure of water activity which is a thermodynamic description of water in a system (relative to pure water) which is controlled by different parameters e.g. the colligative effects of dissolved species, sometimes referred to as solutes which can be proteins and excipients. Sometimes water activity is explained as a parameter describing the tendency of water molecules to “escape” the system, compared to tendency of water molecules to escape pure water. Addition of solutes reduce the escaping tendency (increases the osmolality). Osmolality is a measure of water activity, hence osmolality describes water (and not the solutes) and is affected by solutes. Osmolality can be measured by water dew point depression (vapor pressure osmometry) and by water freezing point depression. A explained desired levels of osmolality can be achieved by the addition of excipients such as buffers, such as salts e.g. NaCl, arginine-HCl, such as amino acids including, but not limited to, histidine, glycine, arginine, methionine, and proline, sugars or sugar alcohols including, but not limited to, mannitol, trehalose, sucrose. Excipients can have the effect of both modifying osmolality and being a protein stabilizer. Additional stabilizers and tonicity agents that are suitable for adjusting osmolality are described in references such as the handbook of Pharmaceutical Excipients (Fourth Edition, Royal Pharmaceutical Society of Great Britain, Science & Practice Publishers) or Remingtons: The Science and Practice of Pharmacy (Nineteenth Edition, Mack Publishing Company). In the context of the present disclosure the terms “isoosmotic,” “isotonic,” “substantially isosmotic,” and “substantially isotonic” are used interchangeably to refer to formulations having an osmolality ranging from about 270 mOsm/kg to about 450 mOsm/kg. In embodiments to about 380 mOsm/kg, or from about 270 mOsm/kg to about 370 mOsm/kg, or from about 300 mOsm/kg to about 330 mOsm/kg.

Embodiments of present formulations include those that are isotonic or near isotonic and have an osmolality range of about 250 to 450 mOsm/kg or 275 to 325 mOsm/kg, including an osmolality of 250, 251, 252, 253, 254, 255, 256, 257, 258, 259, 260, 261, 262, 263, 264, 265, 266, 267, 268, 269,270, 271, 272, 273, 274, 275, 276, 277, 278, 279, 280, 281, 282, 283, 284, 285, 286, 287, 288,289, 290, 291, 292, 293, 294, 295, 296, 297, 298, 299, 300, 301, 302, 303, 304, 305, 306, 307,308, 309, 310, 311, 312, 313, 314, 315, 316, 317, 318, 319, 320, 321, 322, 323, 324, 325, 326,327, 328, 329, 330, 331, 332, 334, 335, 336, 337, 338, 339, 340, 341, 342, 343, 344, 345, 346,347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384,385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 516, 417, 418, 419, 420, 421, 422, 423, 424, 425, 526, 427, 428, 429, 430, 431, 432, 433, 434, 435, 436, 437, 438, 439, 440, 441, 442, 443, 444, 445, 446, 447, 448, 449, 450 mOsm/kg.

Embodiments of antibody formulations include those described herein that have low viscosity despite having a high concentration of antibody in solution. Embodiments of present antibody formulations, formulations having a viscosity of below 25 cP at 25° C. Embodiments of formulations, having a viscosity of between 10 and 20 cP at 25° C. Embodiments include formulations having a viscosity of 12-15 cP at 25° C. It is understood that these ranges and values are not limited to the enumerated numbers and includes further fractional increments.

Embodiments include pharmaceutical containers comprising a vessel and a pharmaceutical formulation as disclosed herein. A vessel is something that holds the pharmaceutical formulation of the invention and can be any suitable vessel known in the art, including, but not limited to a vial, bottle, syringe, or any of a variety of formats well known in the art for packaging pharmaceutical formulations, including subcutaneous and transdermal delivery devices. The syringe may be filled with a pharmaceutical formulation of as disclosed herein prior to distribution to end users (i.e. “prefilled syringe”). Embodiments of the invention include a prefilled syringe containing a pharmaceutical formulation as disclosed herein, wherein the prefilled syringe is in the form of an “autoinjector,” Embodiments of the invention include a prefilled syringe containing the formulations as disclosed herein in the form of an “autoinjector”. Examples of suitable pen and autoinjector delivery devices include, but are not limited to companies like Ypsomed such as “Ypsomate2.25” and “YpsomatePro”; SHL group such as “Molly”; Owen Mumford such as “Aidaptus” or BD such as “Intevia”

In order for the formulations to be used for in vivo administration, they must be sterile. The formulation may be rendered sterile by filtration through sterile filtration membranes. The therapeutic compositions herein is preferable formulated in a single use, prefilled device or autoinjector. The route of administration is in accordance with known and accepted methods, such as by single or multiple injections by subcutaneous administration.

Buffers are used to control the pH in a range which optimizes the therapeutic effectiveness, especially if stability is pH dependent. Buffers are preferably present at concentrations ranging from about 5 mM to about 50 mM. Suitable buffering agents for use with the present invention include both organic and inorganic acids and salts thereof as well as amino acids. For example histidine, citrate, phosphate, succinate, tartrate, fumarate, gluconate, oxalate, lactate, acetate, trimethylamine salts such as Tris.

According to experiments conducted with the current antibody, pH should preferably be within 5.5 and 6.5. In embodiments of the invention the stability of the compound is optimal at pH of about 6.0. In embodiments of the invention the formulations have a pH of about 5.5-6.5. In embodiments this means any of the values within the range. Examples are 5.5, 5.6, 5.7, 5.8, 5.9, 6.0, 6.1, 6.2, 6.3, 6.4 and 6.5. In embodiments the PH is about 6.0.

In embodiments of the invention the buffering agent used in the antibody formulation is histidine, which may be at a concentration of about 5-35 mM. In some examples, the histidine is at a concentration of about 10-30 mM, 15-25 mM, In specific examples, the histidine is at a concentration of about 20 mM.

Stabilizers are present to adjust or maintain the stability of a protein in the formulation. When stabilizers are used with large biomolecules such as proteins including antibodies, dependent on their specific character, these can interact with the charged groups as well as with the hydrophilic groups and the hydrophobic groups of the amino acid side chains, as well as with hydrophobic patches on the surface of the protein and thereby decreasing the likelihood of unwanted intermolecular interactions (protein-protein interactions). Stabilizers can also by preferential exclusion from protein surfaces decreased the tendency of protein unfolding/structural degradation. Stabilizers can also increase the chemical stability of proteins.

Stabilizers can be present in any amount taking into account the amounts of the other ingredients and the osmolality limits. Stabilizers include polyhydric sugar alcohols, preferably trihydric or higher sugar alcohols, such as glycerin, erythritol, arabitol, xylitol, sorbitol and mannitol. Other typical stabilizers include: salts such as NaCl, amino acids such as alanine, glycine, glutamine, asparagine, histidine, methionine, arginine, lysine, ornithine, leucine, 2-phenylalanine, glutamic acid, threonine, proline etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol, hydrophilic polymers such as polyvinylpyrrolidone; monosaccharides (e.g., xylose, mannose, fructose, glucose; disaccharides (e.g., lactose, maltose, sucrose, trehalose); trisaccharides such as raffinose; and polysaccharides such as dextrin or dextran.

The present invention has been investigated with respect to optimal use of stabilizers in the formulation. In embodiments of the invention the formulations of the present invention are substantially free of NaCl. NaCl is normally a widely used stabilizer and tonicity agent, that is used for example to adjust osmolality, increase stability and reduce viscosity. However, in the present antibody formulation presence of NaCl destabilized the antibody.

The formulations of the present invention showed temperature dependent stability variation. When tested at high temperature conditions such as around 50° C. the formulations containing NaCl, arginine and/or aspartic acid in general contained higher amounts of protein aggregates after heat exposure.

Also at lower temperatures, such as below 40° C. there were higher amounts of protein aggregates. In general, there seemed to be a larger amount of aggregates of the antibody after heat exposure where arginine and/or aspartic acid were present. These experiments suggested that arginine and/or aspartic acid should be avoided in the formulation.

In formulations exposed to high temperature, such as up to 40° C., containing sucrose, there was less protein aggregates, which indicated a stabilizing effect of sucrose, and also an improved stabilizing effect of sucrose over for example mannitol.

In formulations exposed to high temperature, such as up to 40° C., there was an indication that the stabilizing effect was proline>glycine>methionine. Experiments measuring viscosity however showed that proline containing formulations had higher viscosity.

When sucrose is present in the formulations, it appears across all experiments that low levels of aggregates are detected.

To meet the requirements related to viscosity, different experiments were conducted to investigate the viscosity of the formulation, in particular the effect of excipients on viscosity was investigated and in particular the effect of antibody concentration on viscosity was investigated. Different viscosity lowering agents can be used and tested to meet the requirements set for the viscosity. Stabilization of formulations is often tested under “accelerated conditions” for example using high temperatures to accelerate protein degradation and hence to accelerate the experimental progress of formulation development. Temperatures of up to 50° C. can be used, or up to 40° C.

In such experiments, arginine, which is considered a viscosity lowering agent, was not among the desired compounds due to the experiments, as described above, showing that arginine had a destabilizing effect on the antibody. These data showed that the antibody could be stabilized by sucrose. However, in one experiment sucrose containing formulations were observed to change color. It was therefore tested to substitute sucrose with trehalose. For different sets of formulation it was found that substitution of sucrose with trehalose was associated with lower aggregation levels after storage at 40° C.

Although experimental data clearly showed that arginine increased the aggregation tendency, arginine was included in further experiments.

After changing the temperature in the stability testing, it appeared that the destabilizing effect of arginine could be prevented by keeping the temperature below 30° C. When these conditions were applied trehalose in combination with arginine showed the best stabilizing effect in terms of limiting protein aggregation. Arginine was surprisingly having a stabilizing effect when experiments were conducted at 5-25° C.

Particularly for formulations with lower concentrations of the antibody such as about 150 mg/mL, the viscosity lowering agent is about 0 mM, whereas at higher concentrations of the antibody the viscosity lowering agent (in particular arginine) is present at concentrations around 75-100 mM in the formulation in addition to the other amino acids present. The concentration of each of the excipients must meet the overall criteria for formulation osmolality.

For high concentrations of the antibody in the formulation, for example 175-225 mg/ml, the viscosity lowering agent must be present at a suitable concentration.

For lower concentrations of the antibody, for example 150-175 mg/mL, the viscosity of the formulation is lower, and therefore addition of viscosity reducing compounds may be either unnecessary or in a low amount.

In embodiments of the invention the formulation according to the present invention comprises the IL-22R antibody, a suitable buffer, an antioxidant, one or more suitable stabilizer(s) and a non-ionic surfactant and optionally a viscosity modifier. In embodiments the PH is of about 5.5-6.5. In embodiments the PH is 6.0. In embodiments the buffer is a histidine buffer. In embodiments the formulation contains glycine. In embodiments glycine is present in up to 80 mM. In other embodiments the formulation contains arginine. In embodiments arginine is present in up to 100 mM. In embodiments the antibody is present in 135-175 mg/mL and glycine is present 80 mM. In embodiments the antibody is present in 175-225 mg/mL and arginine is present in 80 mM.

Non-ionic surfactants or detergents are present to prevent surface adsorption and to help solubilize the therapeutic protein as well as to protect the therapeutic protein against agitation induced aggregation as well as against shear surface stress. Hence non-ionic surfactants can stabilize without causing denaturation of the active therapeutic protein or antibody (in contrast for example to an ionic surfactant). A non-ionic surfactant is a type of surfactant that does not carry a charge on its hydrophilic head group and therefore has no net electrical charge. Non-ionic surfactants are present in a range of about 0 to about 2 mg/ml, 0.05 mg/ml to about 1.0 mg/ml, preferably about 0.07 mg/ml to about 0.2 mg/ml. Suitable non-ionic surfactants include polysorbates (20, 40, 60, 65, 80, etc.), polyoxamers (184, 188, etc.), Pluronic® polyols (poloxamer 188), Triton®, polyoxyethylene sorbitan monoethers (Tween®-20 (polysorbate 20), Tween®-80 (polysorbate 80), etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose.

Suitably the non-ionic surfactant is polysorbate 20, polysorbate 80 or poloxamer 188. In embodiments the non-ionic surfactant is present at a concentration of about 0.005-1% (w/w). In embodiments the concentration of the non-ionic surfactant such as polysorbate 20 may range from 0.005-0.5% (w/w). In embodiments the non-ionic surfactant is polysorbate 20 at a concentration of about 0.01-0.08% (w/w). In embodiments the non-ionic surfactant is polysorbate 20 at a concentration of about 0.01-0.03% (w/w). In embodiments the concentration of polysorbate 20 is 0.02% (w/w).

Formulations of antibodies may be further stabilized by addition of an anti-oxidant. In experiments conducted with the antibody of the present invention, Methionine oxidation was detected at four methionie residues in the Anti IL22R heavy chain (HC): M255 (most suseptable to oxidation), M34, M83 and M431 (second most suspetable to oxidation).

The data is shown as total increase in met oxidation for all four positions in table below. The data show that methionine added to the formulation can limit methionine residue oxidation. Data supports selection of methionine in formulation at a concentration >5 mM. The results show that antioxidant such as methionine can be present in a concentration of 5-30 mM. In embodiments of the invention antioxidant is present between 10-25 mM. In embodiments of the invention antioxidant is present between 15-25 mM. In embodiments of the invention antioxidant is present between 17-23 mM. In some embodiments of the invention about 20 mM. In embodiments the methionine in formulation at a concentration >5 mM

The anti-IL22R antibody of the present formulation may be present at a concentration of about 135 mg/mL to about 250 mg/mL. In embodiments, the antibody is present in a concentration of about 150 mg/ml to about 225 mg/mL. In embodiments, the antibody is present in a concentration of about 140 mg/ml to about 180 mg/mL. For embodiments of the invention this means that the antibody is present in 150 mg/ml to 200 mg/mL, or from 175 mg/ml to 200 mg/mL, or from 150 mg/mL to 175 mg/mL, or at about 150 mg/mL, or at about 175 mg/mL, or at about 200 mg/ml or at about 225 mg/mL.

Dosage regimens and therapeutic application:

The present invention covers a range of antibody concentrations. The formulations meet the criteria set up. The formulations are useful in treating diseases responsive to anti-IL-22R treatment.

To practice the method disclosed herein, an effective amount of any of the IL-22R antibody formulations disclosed herein can be administered to a subject (e.g., a human) in need of the treatment via a suitable route, such as subcutaneous injection or intramuscular injection. The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a disorder associated with IL-22R. Exemplary IL22R-associated disorders include, but are not limited to inflammatory diseases such as psoriasis, psoriatic arthritis, contact dermatitis and atopic dermatitis. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Frequency, number and volume of administration may be determined and adjusted over the course of therapy, and is generally, but not necessarily, based on treatment and/or suppression and/or amelioration and/or delay of a disorder associated with IL-22R. For the purpose of the present disclosure, the appropriate dosage of an IL22R antibody will depend on the specific IL-22R antibody(s) (or compositions thereof) employed, the type and severity of disorder associated with IL-22R, whether the antibody is administered for preventive or therapeutic purposes, previous therapy, the patient's clinical history and response to the antibody, and the discretion of the attending physician. As used herein, the term “treating” refers to the application or administration of a composition including one or more active agents to a subject, who has a disease associated with IL-22R, with the purpose to cure, heal, alleviate, relieve, alter, remedy, ameliorate, improve, or affect the disorder, the symptom of the disease, or the predisposition toward the disease. To perform the methods as described herein, any of the anti-IL-22R antibodies may be given to a subject in need of the treatment (e.g., a human patient) by a single dose or by multiple doses via a suitable route, for example, subcutaneous injection. The dosage of the anti-IL-22 antibody may range from about 150 mg/mL in one, two or three injections of for example 1 mL, 1.5 mL, 2 mL, 2.5 mL or 3 mL to about 200 mg/mL in one, two or three injections of for example 1 mL, 1.5 mL, 2 mL, 2.5 mL or 3 mL. The administration of an IL-22R antibody may be a single treatment or a repeated administration over a preselected period of time in a series of spaced doses.

### EXAMPLES

All anti IL22R samples/formulations used to generate data for the present examples were prepared with milliQ water or water for injection (WFI). All pH measurement were conducted at room temperature with calibrated equipment. The IL 22 receptor antibody used in the examples below is the antibody defined by SEQ ID No 1, 2, 3, 4, 5, 6, 7, 8, 9 and 10.

### Example 1: NaCl Cannot be Used as Tonicity Agent Due to it Destabilizing Effect on Anti IL22R

NaCl, a well-known tonicity agent (or osmolality regulating agent), cannot be used as tonicity agent for anti-IL22R due to its destabilizing effects (increased aggregation) on this antibody. Sample preparation: anti-IL22R was buffer-exchanged into 20 mM histidine pH 6.5 and the concentration was adjusted to 40 mg/mL. The protein solution was diluted 1:1 with different NaCl solutions (NaCl in milliQ water). The final anti-IL22R samples for stability evaluation contained 20 mg/mL anti-IL22R, 10 mM histidine, and NaCl at concentrations: 0 mM, 25 mM, 50 mM, 100 mM, 150 mM and 300 mM

Analyses: Protein samples were analysed by SEC (size exclusion chromatography) before and after heat stress using SEC column: Waters BEH 200 SEC, 300 mm×4.6 mm column. Column temperature 25° C. Mobile phase: 100 mM Sodium Phosphate Monobasic Monohydrate and 200 mM Sodium Chloride (NaCl). Flow rate: 0.15 ml/min. Detection 280 nm and 215 nm. Run time 30 minutes. Integration procedure: HMWP % (total area percent of peaks eluting before the monomer peak), LMWP % (total area percent of peaks eluting after the monomer peak). Samples were analysed just after preparation as well as after 30 minutes at 60° C. and 1 hour at 60° C. The SEC data clearly shows correlation between NaCl concentration and decreased stability (see table of SEC integration data below). At t=0 no chromatographic differences between samples were observed, whereas significant differences were observed after heat exposure.

Results: Monomer loss during heat exposure is more pronounced in the presence of NaCl. The monomer loss is caused by protein aggregation, detected as HMWP in the SEC chromatograms. No significant changes in LMWP were observed.

| | mM NaCl | 0.00 | 25 mM | 50 mM | 100 mM | 150 mM | 300 mM |
|---|---|---|---|---|---|---|---|
| HMWP % | t = 0 | 1.75 | 1.74 | 1.71 | 1.70 | 1.70 | 1.69 |
| | 0.5 hour | 9.08 | 10.57 | 13.20 | 17.48 | 24.82 | 26.71 |
| | @60° C. | | | | | | |
| | 1 hour | 17.75 | 26.03 | 26.62 | 31.16 | 44.95 | 50.07 |
| | @60° C. | | | | | | |
| Monomer % | t = 0 | 96.92 | 97.04 | 97.06 | 97.09 | 97.07 | 97.07 |
| | 0.5 hour | 89.56 | 88.07 | 85.47 | 81.07 | 73.68 | 71.74 |
| | @60° C. | | | | | | |
| | 1 hour | 81.10 | 72.61 | 71.99 | 67.41 | 53.74 | 48.66 |
| | @60° C. | | | | | | |
| LMWP % | t = 0 | 1.34 | 1.23 | 1.24 | 1.22 | 1.24 | 1.24 |
| | 0.5 hour | 1.37 | 1.37 | 1.34 | 1.45 | 1.50 | 1.57 |
| | @60° C. | | | | | | |
| | 1 hour | 1.16 | 1.35 | 1.40 | 1.43 | 1.32 | 1.28 |
| | @60° C. | | | | | | |

### Example 2: Thermal Stability of Anti-IL22R is Affected by pH (Higher at Increasing pH)

Sample preparation: anti-IL22R was buffer-exchanged into 20 mM histidine pH 6.0 and the concentration was adjusted to 2 mg/mL. The protein solution was diluted 1:1 in pH screening-solutions with respective pH values: 3.2, 3.5, 5, 6, 7, 8, 9 and 10. The pH screening solutions contained one of the following two buffers or a mixture of the following two buffers 1) 100 mM histidine, 100 mM glycylglycine, 100 mM Na-acetate pH 10 and 2) 100 mM histidine, 100 mM glycylglycine, 100 mM acetic acid pH 3.2. The final anti-IL22R samples for stability evaluation contained 1 mg/mL anti-IL22R, 60 mM histidine, 50 mM glycylglycine, 50 mM Na-acetate/acetic acid. The sample pH was measured to: pH 3.34, pH 3.57, pH 5.09, pH 5.96, pH 6.79, pH 7.64, pH 8.78, pH 9.48.

Analyses: Samples were exposed to increasing temperature 25-85° C. to induce protein aggregation. The aggregation onset temperature differs between proteins and differs according to pH and sample composition for each protein. If a protein can tolerate high temperature before it starts aggregating, it is regarded thermally stable. Aggregation onset temperatures were determined for anti-IL22R at different pH values. The samples were analysed by Dynamic- and Static Light Scattering (DLS and SLS) on Wyatt Platereader III, Software: DYNAMICS version 7.8.2.18, using 2×35 uL per sample in Aurora 384 well plate ABA2-10100A. Light scattering data was obtained during heating from 25-85° C. The aggregation onset temperatures (Tagg values) were determined from the light scattering data vs temperature curves. For each sample Tagg was determined from three different data vs temperature traces by the equipment software 1) Tagg from the estimated hydrodynamic radius vs temperature trace, by DLS 2) Tagg from the estimated molecular weight vs temperature trace, by SLS 3) Tagg from the normalized scattering intensity, by SLS. The difference between the estimated Tagg values is shown in the result table below.

Results: Sample pH affects the thermal stability of anti-IL22R. The antibody is less stable at low pH. The highest Tagg values were detected for the anti-IL22R samples at pH 6.8-8.8, and highest at pH 7.6

| samples pH | 3.3 | 3.6 | 5.1 | 6.0 | 6.8 | 7.6 | 8.8 | 9.5 |
|---|---|---|---|---|---|---|---|---|
| Tagg based on Rh | 40.7 | 43.5 | 52.9 | 57.5 | 60.1 | 60.7 | 60.2 | 59.1 |
| estimation (DLS) | | | | | | | | |
| Tagg based on MW | 38.7 | 44.8 | 47.8 | 55.6 | 58.7 | 59.9 | 59.0 | 57.6 |
| estimation (SLS) | | | | | | | | |
| Tagg based on NSI (SLS) | 38.6 | 42.4 | 51.9 | 56.2 | 59.2 | 59.9 | 59.7 | 58.8 |

### Example 3: Stability of Anti-IL22R is Affected by pH, Especially Fragmentation is Increased at Increasing pH

Sample preparation: anti-IL22R was buffer-exchanged into 20 mM histidine pH 6.5 and the concentration was adjusted to 40 mg/mL. The protein solution was diluted 1:1 in pH screening-solutions with respective pH values: pH 5.5, 6.0, 6.5, 7.0, 7.5, 8.0 and 8.5. The pH screening solutions contained a mixture of the following two buffers 1) 100 mM histidine, 100 mM glycylglycine, 100 mM Na-acetate and pH was adjusted to pH 5.5, 6.0, 6.5, 7.0, 7.5, 8.0 and 8.5. The samples were analysed weekly during 3 weeks at 30° C.

Analyses: Protein samples were analysed by SEC (size exclusion chromatography) using SEC column: Waters BEH 200 SEC, 300 mm×4.6 mm column. Column temperature 25° C. Mobile phase: 100 mM Sodium Phosphate Monobasic Monohydrate and 200 mM Sodium Chloride (NaCl). Flow rate: 0.15 ml/min. Detection 280 nm and 215 nm. SEC integration procedure: HMWP % (total area percent of peaks eluting before the monomer peak), LMWP % (total area percent of peaks eluting after the monomer peak). Samples were analysed just after preparation as well as after 6 days, 7 days, and 14 days and 21 days at 30° C. The SEC data shows correlation between pH and mAb degradation (aggregation/increased HMWP % and fragmentation/increased LMWP % (see table of SEC integration data below).

Results: Sample pH affects aggregation and in particular fragmentation of anti-IL22R at 30° C. At 30° C. the antibody is observed to be less stable at high pH. The highest HMWP and LMWP values are observed at pH 8.5. Under these experimental conditions LMWP formation is most pronounced, whereas protein aggregation (HMWP formation) appears to be relatively slow. The generated stability data shows that, with regards to fragmentation, anti-IL22R is most stable at pH 5.5-7. At pH 7.5-8.5 anti-IL22R is unstable under the present conditions.

To confirm the data anti-IL22R was also analyzed by CE-SDS (Equipment: Maurice from ProteinSimple). A number of experimental challenges were observed such as large variation in MW profiles when comparing replicates (e.g. with regards to retention time and peak shape and peak splitting). However, CE-SDS analyses (data not included) confirmed that anti-IL22R fragmentation is more pronounced at pH >7.

| time | | | | | | | |
|---|---|---|---|---|---|---|---|
| (days) | pH 5.5 | pH 6 | pH 6.5 | pH 7 | pH 7.5 | pH 8 | pH 8.5 |
| | SEC-HMWP % | | | | | | |
| 0 | 1.21 | 1.21 | 1.23 | 1.23 | 1.22 | 1.19 | 1.16 |
| 6 | 0.85 | 0.89 | 0.91 | 0.88 | 0.97 | 1.12 | 1.27 |
| 7 | 0.87 | 0.86 | 0.90 | 0.91 | 1.02 | 1.12 | 1.38 |
| 14 | 0.87 | 0.85 | 0.92 | 0.93 | 1.11 | 1.31 | 1.69 |
| 21 | 0.86 | 0.87 | 0.76 | 1.02 | 1.20 | 1.60 | 2.49 |
| | SEC-LMWP % | | | | | | |
| 0 | 1.11 | 1.07 | 1.09 | 1.09 | 1.13 | 1.13 | 1.10 |
| 6 | 1.16 | 1.25 | 1.19 | 1.20 | 1.29 | 1.64 | 2.69 |
| 7 | 1.24 | 1.25 | 1.27 | 1.30 | 1.36 | 1.78 | 3.26 |
| 14 | 1.66 | 1.76 | 1.72 | 1.76 | 2.09 | 2.66 | 6.08 |
| 21 | 3.32 | 3.18 | 3.36 | 3.38 | 3.83 | 5.56 | 10.87 |

Comparison of data in examples 2 and 3 shows that changing pH can have opposing effects on different aspects of anti IL22R stability.

### Example 4: Chemical Degradation of Anti-IL22R is Increased at Increasing pH

Sample preparation: anti-IL22R was buffer-exchanged into 20 mM histidine pH 6.5 and the concentration was adjusted to 40 mg/mL. The protein solution was diluted 1:1 in pH screening-solutions with respective pH values: pH 5.5, 6.0, 6.5, 7.0, 7.5, 8.0 and 8.5. The pH screening solutions contained a mixture of the following two buffers 1) 100 mM histidine, 100 mM glycylglycine, 100 mM Na-acetate and pH was adjusted to pH 5.5, 6.0, 6.5, 7.0, 7.5, 8.0 and 8.5. The samples were analysed weekly during 2 weeks at 30° C.

Analyses: Protein samples were analysed by CIEX (Cat Ion Exchange chromatography) using CIEX column: MabPac SCX-10 RS 50×2.1 mm, 5 μm. Column temperature 25° C. Mobile phase A: Diluted CX-1 pH gradient buffer A pH 5.6, from Thermo Scientific (10× diluted in milliQ water). Mobile phase B: Diluted CX-1 pH gradient buffer B PH 10.2, from Thermo Scientific (10× diluted in milliQ water). Flow rate: 0.4 mL/min Detection 280 nm and 215 nm. Run time 23 minutes. Gradient: (Initial: 10% B held at 2 min. Sample separation: Linear gradient 10-40% B over 13 min. Change mobile phase to 90% B hold for 3 minutes. Returned to the starting solvent 10% B hold for 5 min. CIEX integration procedure: Acidic peaks % (total area percent of peaks eluting before the main charge variant), Basic peaks % (total area percent of peaks eluting after the main charge variant). Increase in acidic peaks (was shown by MS peptide mapping to be related to anti-IL22R deamidation) is quantified as (Acidic peaks % @tₓ-Acidic peaks % @t₀). For this mAb the percentage of basic peak area is observed to decrease during stability studies. Hence, the total chemical change over time is quantified as the increase in acidic peak plus the numerical decrease in basic peaks.

Results: Sample pH affects chemical degradation of anti-IL22R at 30° C. At 30° C. the chemical stability of anti-IL22R was observed to gradually decrease with increasing pH. The chemical quantified as the increase in acidic peak area %+the decrease in basic peak area % is shown in table below

Increase in acidic peak area %+decrease in basic peak area % (during 6 days and 2 weeks at 30° C.) at pH 5.5, 6.0, 6.5, 7.0, 7.5, 8.0 and 8.5

| pH | 5.5 | 6 | 6.5 | 7 | 7.5 | 8 | 8.5 |
|---|---|---|---|---|---|---|---|
| 6 d at | 0.3 | 0.6 | 2.2 | 4.2 | 8.0 | 13.3 | 23.7 |
| 30 C. | | | | | | | |
| 2 w at | 1.7 | 5.7 | 6.5 | 11.3 | 21.5 | 30.9 | 42.8 |
| 30 C. | | | | | | | |

The increase in acidic peaks alone (table below) alone, suggests that pH 6 provides the highest stability during 2 weeks at 30° C.

Increase in acidic peak area % (during 6 days and 2 weeks at 30° C.) at pH 5.5, 6.0, 6.5, 7.0, 7.5, 8.0 and 8.5

| pH | 5.5 | 6 | 6.5 | 7 | 7.5 | 8 | 8.5 |
|---|---|---|---|---|---|---|---|
| 6 d at | 0.0 | −0.4 | 1.6 | 2.0 | 5.1 | 9.0 | 16.0 |
| 30 C. | | | | | | | |
| 2 w at | 1.3 | 0.4 | 4.5 | 7.1 | 13.6 | 20.8 | 31.4 |
| 30 C. | | | | | | | |

Data presented in example 2, 3 and 4 suggests anti IL22R to be most stable at pH 5.5-6.0

### Example 5: Anti IL22R Stabilization and Destabilization by Excipients

The influence of excipients on anti-IL22R denaturation— and aggregation tendency. Fourteen excipients of various types were tested in this study: Sucrose, trehalose, maltose and lactose (disaccharides of which sucrose and trehalose are regarded as nonreducing sugars and hence most suitable for development of pharmaceutical liquid protein formulations), histidine, proline, lysine, glycine, glutamic acid (amino acids), mannitol, sorbitol (polyols), sodium phosphate and NaCl (slats), succinic acid.

Sample preparation: anti-IL22R was buffer-exchanged and concentrated to 56.5 mg/ml in 80 mM histidine pH 5.98 and the concentration was adjusted to 40.3 mg/ml in in 80 mM histidine pH 5.99. Excipients stock solutions were prepared, and pH adjusted to pH 6±0.1. The protein solution was diluted four times in excipient stock solution (75 uL protein solution±225 uL excipient stock solution). The composition of the final protein solutions is shown in table below.

The samples were analysed weekly during 2 weeks at 30° C.

10 mg/mL anti-IL22R, 20 mM
histidine, pH 6
10 mg/mL anti-IL22R, 100 mM
histidine, pH 6
10 mg/mL anti-IL22R, 100 mM lysine,
pH 6
10 mg/mL anti-IL22R, 100 mM
proline, pH 6
10 mg/mL anti-IL22R, 100 mM
glycine, pH 6
10 mg/mL anti-IL22R, 100 mM
glutamic acid/glutamate, pH 6
10 mg/mL anti-IL22R, 100 mM
succinic acid/succinate, pH 6
10 mg/mL anti-IL22R, 150 mM sodium
phosphate, pH 6
10 mg/mL anti-IL22R, 150 mM sodium
chloride, pH 6
10 mg/mL anti-IL22R, 300 mM
mannitol, pH 6
10 mg/mL anti-IL22R, 300 mM
sorbitol, pH 6
10 mg/mL anti-IL22R, 300 mM
sucrose, pH 6
10 mg/mL anti-IL22R, 300 mM
lactose, pH 6
10 mg/mL anti-IL22R, 300 mM
maltose, pH 6
10 mg/ml anti-IL22R, 300 mM
trehalose, pH 6

Analyses: Protein samples were analysed by SEC (size exclusion chromatography) using SEC column: Waters BEH 200 SEC, 300 mm×4.6 mm column. Column temperature 25° C. Mobile phase: 100 mM Sodium Phosphate Monobasic Monohydrate and 200 mM Sodium Chloride (NaCl).

Flow rate: 0.15 ml/min. Detection 280 nm. SEC integration procedure: HMWP % (total area percent of peaks eluting before the monomer peak). Samples were analysed after preparation as well as after exposure to elevated temperatures (40° C. for 4 weeks as well as 50° C. for 5 hours, 2 days and 3 days) as indicated by data table below. The SEC data shows how anti-IL22R aggregation (HMWP formation) is affected by different excipients.

The denaturation temperature of anti-IL22R in the different samples was analysed by intrinsic fluorescence detection during heating scans, using Uncle from Unchained Labs. The denaturation temperature (Td) was determined from the inflection point in the fluorescence vs temperature curve. Higher Td means higher thermal stability.

Results: The denaturation data (first data column in table below) shows that 100 mM histidine, 100 mM lysine, 100 mM glutamic acid, 100 mM succinate, 150 mM sodium phosphate, 150 mM sodium chloride reduces the denaturation temperature anti-IL22R, hence destabilizing in terms of increasing the tendency of temperature induced denaturation. The same excipients were observed to increase the aggregate content in the samples when exposed to thermal stress: 2 and 3 days at 50° C. as well as 4 weeks at 40° C.

The SEC data indicates (comparison after 4 weeks at 40° C.) a stabilizing effect of the polyols (mannitol and sorbitol) and the disaccharides (sucrose, lactose, maltose and trehalose). 300 mM trehalose and mannitol furthermore increase the denaturation temperature of anti-IL22R.

| | | | HMWP % | HMWP % | HMWP % | HMWP % |
|---|---|---|---|---|---|---|
| | Td | HMWP % | (5 h | (2 d | (3 d | (4 w |
| | (° C.) | (t = 0) | 50° C.) | 50° C.) | 50° C.) | 40° C.) |
| ref | 66.1 | 0.61 | 0.45 | 0.44 | 0.89 | 1.27 |
| histidine | 60.8 | 0.95 | 0.75 | 0.62 | 2.82 | 1.58 |
| proline | 65.5 | 0.56 | 0.45 | 0.4 | 0.81 | 1.25 |
| lysine | 57.0 | 0.94 | 0.85 | 1.73 | 12.66 | 2.99 |
| glycine | 65.7 | 0.60 | 0.43 | 0.52 | 0.86 | 1.47 |
| glutamic | 60.6 | 0.74 | 0.5 | 0.7 | 2.13 | 1.71 |
| acid | | | | | | |
| succinic | 59.4 | 0.74 | 0.83 | 1.28 | 4.8 | 1.22 |
| acid | | | | | | |
| Na- | 65.0 | 0.69 | 0.52 | 0.97 | 3.54 | 1.61 |
| phosph | | | | | | |
| NaCl | 56.6 | 0.89 | 0.91 | 0.97 | 10.17 | 1.65 |
| mannitol | 66.5 | 0.70 | 0.48 | 0.56 | 0.89 | 0.73 |
| sorbitol | 66.2 | 0.87 | 0.46 | 0.47 | 0.83 | 0.72 |
| sucrose | 66.1 | 0.79 | 0.44 | 0.47 | 0.83 | 0.74 |
| lactose | 66.2 | 0.67 | 0.44 | 0.54 | 0.73 | 0.75 |
| maltose | 66.1 | 0.65 | 0.43 | 0.48 | 0.81 | 0.99 |
| trehalose | 67.1 | 0.78 | 0.45 | 0.46 | 0.85 | 1.05 |

### Example 6: Anti IL22R Stabilization and Destabilization by Excipients

The influence of excipients on anti-IL22R aggregation at protein concentration 65-66 mg/mL. During sample preparation, buffer exchange and concentration in Amicon Ultra-4 mL (30K) spin filters, centrifugation 4000G at room temperature an important observation was made: Significant protein loss (anti-IL22R loss) was observed when concentrated from a test batch with 9.49 mg/ml anti IL22R in 80 mM sodium phosphate, 40 mM sodium chloride, pH 7, whereas no protein lost was observed when concentration of anti-IL22R was performed in 20 mM histidine pH 6.5. So, the unfavorable effects of sodium chloride and sodium phosphate (such as decreased thermal stability and increased aggregation tendency which is described in previous examples) also include increased surface adsorption during handling and formulation processes.

The samples to be investigated further in this study were prepared by buffer exchange and concentrated into 9 different formulations described in table below. Hereafter the protein concentration was adjusted to 65-66 mg/ml to make all samples equal with respect to anti-IL22R concentration.

Analyses: Protein samples were analysed by SEC (size exclusion chromatography) using SEC column: Waters BEH 200 SEC, 300 mm×4.6 mm column. Column temperature 25° C. Mobile phase: 100 mM Sodium Phosphate Monobasic Monohydrate and 200 mM Sodium Chloride (NaCl).

Flow rate: 0.15 ml/min. Detection 280 nm. SEC integration procedure: HMWP % (total area percent of peaks eluting before the monomer peak). Samples were analysed after preparation as well as after exposure to elevated temperatures (40° C. for 4 weeks as well as 50° C. for 5 hours, 2 days and 3 days) as indicated by data table below. The SEC data shows how anti-IL22R aggregation (HMWP formation) is affected by different excipients.

Results: The SEC data in this experiment indicates destabilizing effects of arginine, aspartic acid, combination of arginine and aspartic acid as well as of sodium chloride when the anti-IL22R samples are stored at 50° C. (see SEC-HMWP data in the table below). Arginine and NaCl showed a clear destabilizing effect when anti IL22R was stored 4 weeks at 40° C. In contrast aspartic acid as well as a combination of arginine and aspartic acid was not associated with destabilization (increased aggregation) upon 4 weeks storage at 40° C. Proline, glycine, mannitol, and sucrose has a stabilizing effect (reduced aggregation) on anti-IL22R at both 40° C. and 50° C., as shown by SEC-HMWP data below.

| | SEC-HMWP % | SEC-HMWP % | SEC-HMWP % |
|---|---|---|---|
| | t = 0 | 3 d@50° C. | 4 w@40° C. |
| Ref. 20 mM histidine, pH 5.99 | 2.1 | 6.4 | 7.9 |
| Arginine 200 mM, 20 mM histidine, pH 6.03 | 1.4 | 65.4 | 14.9 |
| Aspartic acid 200 mM, 20 mM histidine, pH 6.07 | 1.4 | 32.2 | 6.1 |
| Arg 100 mM + Asp 100 mM, 20 mM histidine, pH 5.99 | 1.7 | 36.9 | 4.8 |
| Proline 200 mM, 20 mM histidine, pH 6.01 | 1.5 | 3.6 | 4.4 |
| Glycine 200 mM, 20 mM histidine, pH 6.08 | 1.6 | 2.8 | 4.0 |
| NaCl 150 mM, 20 mM histidine, pH 6.01 | 1.3 | 18.5 | 22.2 |
| Mannitol 200 mM, 20 mM histidine, pH 5.97 | 1.5 | 2.5 | 2.7 |
| Sucrose 200 mM, 20 mM histidine, pH 6.04 | 1.7 | 2.3 | 2.9 |

### Example 7: Influence of Excipients on Anti-IL22R Viscosity at Protein Concentration 65-66 mg/mL

Sample preparation: Buffer exchange and concentration in Amicon Ultra-4 mL (30K) spin filters, centrifugation 4000G at room temperature into 9 different formulations described in table below. During sample preparation glycine and arginine were observed to protect against protein loss. The protein concentration was adjusted to 65-66 mg/mL to make all samples equal with respect to anti-IL22R concentration. Viscosity was measured by RheoSence microVisc viscometer at room temperature 22° C.

Results: In general, the viscosity results show relatively small differences between anti-IL22R samples at 65-66 mg/mL. The data may indicate that sucrose, mannitol, and proline at, the tested concentrations, increases the viscosity of the protein samples at 65-66 mg/mL. The influence of excipients was expected to be higher at increased anti IL22R concentration, confirmed in later examples.

| | Viscosity | Difference |
|---|---|---|
| | (mPa*s) | from ref. |
| Ref. 20 mM histidine, pH 5.99 | 1.91 | 0.0 |
| Arginine 200 mM, 20 mM histidine, pH 6.03 | 1.96 | 0.1 |
| Aspartic acid 200 mM, 20 mM histidine, pH 6.07 | 2.04 | 0.1 |
| Arg 100 mM + Asp 100 mM, 20 mM histidine, pH 5.99 | 1.87 | 0.0 |
| Proline 200 mM, 20 mM histidine, pH 6.01 | 2.11 | 0.2 |
| Glycine 200 mM, 20 mM histidine, pH 6.08 | 1.90 | 0.0 |
| NaCl 150 mM, 20 mM histidine, pH 6.01 | 1.98 | 0.1 |
| Mannitol 200 mM, 20 mM histidine, pH 5.97 | 2.18 | 0.3 |
| Sucrose 200 mM, 20 mM histidine, pH 6.04 | 2.52 | 0.6 |

### Example 8: Viscosity as Function of Anti IL22R Concentration

A concentration study was initiated to investigate the impact of anti IL22R concentration on viscosity. Anti IL22R ˜60 mg/mL in 20 mM histidine buffer pH 6.5 was concentrated on an Amicon spin filter (Amicon Ultra 4 mL—MWCo 30K filter), and sampling was performed during concentration about every 5-10 minutes. The concentration increased gradually over time, however more slowly at higher concentrations. Including the initial sample (sample 1) six anti IL22R samples were collected for viscosity measurements. The highest obtained concentration of anti IL22R in these formulations was 214 mg/ml (sample 6), further spinning of the sample did not provide higher protein concentration suggesting that the solubility of anti IL22R at room temperature in 20 mM histidine pH 6.5 is about 210-220 mg/mL. Viscosity was measured by RheoSence microVisc viscometer at room temperature 18-22° C., shear rate of 1400 s⁻¹. The viscosity data show exponential increase in viscosity as function of protein concentration, see table and figure below.

| Sample | #1 | #2 | #3 | #4 | #5 | #6 |
|---|---|---|---|---|---|---|
| Concentration | 61.1 | 95.7 | 132.4 | 179.2 | 193.5 | 214.0 |
| (mg/mL) | | | | | | |
| Viscosity (cP) | 1.7 | 2.8 | 5.6 | 16.5 | 24.4 | 35.7 |

### Example 9: Effects of Anti IL22R on Osmolality

A concentration study was initiated to investigate the impact of anti IL22R on osmolality (osmolality is in theory a measure of the thermodynamic value: water activity). Due to very low molar concentration of protein in solution, the osmolality is expected to be low for a pure protein-water system. Anti IL22R ˜60 mg/mL in 20 mM histidine buffer pH 6.5 was concentrated on Amicon spin filter (Amicon Ultra 4 mL-MWCo 30K filter), and sampling was performed during concentration about every 5-10 minutes. The concentration increased gradually over time, however more slowly at higher concentrations. Including the initial sample (sample 1) six anti IL22R samples were collected for osmolality measurements. The highest obtained concentration of anti IL22R in this formulation was about 214 mg/ml (sample 6), further spinning of the sample did not provide higher protein concentration suggesting that the solubility of anti IL22R in 20 mM histidine pH 6.5 is about 210-220 mg/mL. Osmolality was measured by freezing point osmometry using Osmomat 3000, Gonotec. The osmolality data is shown in table below. Assuming that, 20 mM histidine in these samples contributes to the osmolality with 20 mOsm/kg the data shows that the osmolality contribution from the anti IL22R at concentration 61-214 mg/ml (corresponding to 0.4-1.5 mM) is about 10-22 mOsm/kg. Hence to obtain isotonic formulations excipients need to be added.

| | Concentration (mg/mL) | | | | | | |
|---|---|---|---|---|---|---|---|
| | 61.1 | 95.7 | 132.4 | 179.2 | 193.5 | 213.5 | 213.5 |
| Osmolality | 30 | 33 | 36 | 39 | 42 | 42 | 36 |
| (mOsm/kg) | | | | | | | |

### Example 10 Anti IL22R Stabilization and Destabilization by Excipient Combinations

The influence of excipient combinations on anti IL22R aggregation was evaluated for 32 different anti-IL22R samples. Sample preparation was conducted using GE Helthcare MiniTrap DP 10 Columns and Amicon Ultra-4 mL-MWCO 30K as briefly described below. The target protein concentration was 150 mg/ml. The study ended at having two sets of samples “Low concentration samples” and “High concentration samples” due to initial challenges with preparations of samples at target protein concentration. “Low concentration samples” with 46-59 mg/mL anti IL22R were prepared by procedure A) This procedure resulted in significant protein loss. It was found that protein loss could be avoided by changing the preparation method to procedure B) resulting in the targeted “high concentration samples” with 146-169 mg/mL anti IL22R (samples with protein concentrations >156 mg/mL were diluted to 150 mg/mL, to obtain a samples series with smaller variation in protein concentration. For all samples tween 20 was added after the last step to avoid tween 20 adsorption to columns and filters. For all samples protein concentration was measured by UV280 (by Lunatic from Unchained Labs).

A) Anti IL22R at 60 mg/mL in 20 mM histidine buffer pH 6.5 was concentrated to 150 mg/mL on amicon Ultra-4 mL-MWCO 30K, followed by buffer exchanged on MiniTrap DP 10 Columns from GE Healthcare in to the respective formulations (see table below). This procedure resulted in protein loss and anti IL22R concentrations of 46-59 mg/mL.

B) Anti IL22R at 60 mg/mL in 20 mM histidine buffer pH 6.5 was buffer exchanged on MiniTrap DP 10 Columns from GE Healthcare in to the respective formulations (see table below). After buffer exchange the samples (which in concentration was still around 60 mg/mL) were concentrated to 150 mg/mL on amicon Ultra-4 mL-MWCO 30K. This procedure resulted in anti IL22R samples with 146-169 mg/mL.

*samples were diluted to 150 mg/mL in respective formulations

Analyses: Protein Samples were Analysed by SEC (Size Exclusion Chromatography) Using SEC

| | F1 | F2 | F3 | F4 | F5 | F6 | F7 | F8 | F9 |
|---|---|---|---|---|---|---|---|---|---|
| His | 20 | 20 | 20 | 20 | 20 | 20 | 20 | 20 | 20 |
| (mM) | | | | | | | | | |
| Arg | 50 | 35 | 40 | 47 | 48 | 48 | 55 | | |
| (mM) | | | | | | | | | |
| Asp | | 35 | 40 | 47 | 48 | 48 | | 60 | |
| (mM) | | | | | | | | | |
| Pro | | 35 | 40 | 47 | 48 | | 55 | 60 | 120 |
| (mM) | | | | | | | | | |
| Gly | | 35 | 40 | 47 | | 48 | 55 | 60 | 120 |
| (mM) | | | | | | | | | |
| Met | | 40 | 40 | | 40 | 40 | 40 | 40 | 40 |
| (mM) | | | | | | | | | |
| sucrose | 180 | 35 | | | | | | | |
| (mM) | | | | | | | | | |
| glycerol | | | | | | | | | |
| (mM) | | | | | | | | | |
| mannitol | | | | | | | | | |
| (mM) | | | | | | | | | |
| tween | 0.2 | 0.2 | 0.2 | 0.2 | 0.2 | 0.2 | 0.2 | 0.2 | 0.2 |
| 20 mg/mL | | | | | | | | | |
| pH | 6.0 | 6.0 | 6.0 | 6.0 | 6.0 | 6.0 | 6.0 | 6.0 | 6.0 |
| anti | 56152.7 | 60145.7 | 57147.2 | 59148.3 | 50164.6* | 55151.0 | 52166.4* | 47154.1 | 40159.7* |
| IL22R | | | | | | | | | |
| (mg/mL) | | | | | | | | | |
| anti | | | | | | | | | |
| IL22R | | | | | | | | | |
| (mg/mL) | | | | | | | | | |

| | F10 | F11 | F12 | F13 | F14 | F15 | F16 |
|---|---|---|---|---|---|---|---|
| His | 20 | 20 | 20 | 20 | 20 | 20 | 20 |
| (mM) | | | | | | | |
| Arg | | | | | | | 50 |
| (mM) | | | | | | | |
| Asp | | | | | | | |
| (mM) | | | | | | | |
| Pro | 80 | | 80 | 110 | | | |
| (mM) | | | | | | | |
| Gly | 80 | 80 | | | | | |
| (mM) | | | | | | | |
| Met | 40 | 40 | 40 | | 40 | 40 | |
| (mM) | | | | | | | |
| sucrose | 80 | 180 | 180 | 180 | 70 | 180 | |
| (mM) | | | | | | | |
| glycerol | | | | | 180 | 70 | |
| (mM) | | | | | | | |
| mannitol | | | | | | | 180 |
| (mM) | | | | | | | |
| tween | 0.2 | 0.2 | 0.2 | 0.2 | 0.2 | 0.2 | 0.2 |
| 20 mg/mL | | | | | | | |
| pH | 6.0 | 6.0 | 6.0 | 6.0 | 6.0 | 6.0 | 6.0 |
| anti | 50159.5* | 53168.8* | 53155.7 | 47153.7 | 46160.6* | 52149.5 | 56153.7 |
| IL22R | | | | | | | |
| (mg/mL) | | | | | | | |
| anti | | | | | | | |
| IL22R | | | | | | | |
| (mg/mL) | | | | | | | |

column: Waters BEH 200 SEC, 300 mm×4.6 mm column. Column temperature 25° C. Mobile phase: 100 mM Sodium Phosphate Monobasic Monohydrate and 200 mM Sodium Chloride (NaCl). Flow rate: 0.15 ml/min. Detection 280 nm. SEC integration procedure: HMWP % (total area percent of peaks eluting before the monomer peak). Samples were analysed after preparation as well as after exposure to five freeze/thaw cycles and to high temperature: 50° C. for 3 days. No clear effects of freeze/thaw stress were observed on HMWP levels for any samples, however the SEC data shows how anti-IL22R aggregation (HMWP formation) is affected by excipients. For all practical matters 0.2 mg/ml tween 20 corresponds to 0.02% (w/w) tween 20 (polysorbate 20).

Results: The SEC data in this experiment indicates destabilizing effects of arginine (see SEC-HMWP data in the table below) on anti IL22R (F1-F7). In contrast sucrose seems to stabilize the antibody (F1, F2, F10-F15). Heat induced anti IL22R aggregation was, not surprisingly, found to depend on protein concentration (comparison of third row in the two data tables). Heat induced aggregation in anti IL22R samples containing ˜50 mg/mL was only observed for a few formulations. Following conclusions can be made based on the data generated for the 150 mg/ml samples.
Formulations containing arginine and/or aspartic acid in general contained higher amounts of protein aggregates after heat exposure. For heated samples there seems to be some correlation between arginine concentration and the amount of anti IL22R aggregates (HMWP %), F2-F7 comparison. This is in line with observations presented in example 6.
F1 seems more stable than all other arginine containing formulations, this was assumed to be caused by the stabilizing effect of sucrose at 180 mM.
Comparison of F1 and F16 suggests that sucrose is a better stabilizer than mannitol towards temperature induced aggregation.
Comparison of F4 and F5 suggests glycine to be a better stabilizer than methionine wrt aggregation at high temperature.
Comparison of F5 and F6 suggests proline to be a better anti IL22R stabilizer than glycine.
Aggregation of anti IL22R in F9-F14 was less pronounced compared to all other formulations. These formulations contained different combinations of proline, glycine, methionine, and glycerol. Aggregate content in the F15 sample at high concentration was not determined due to missing SEC injection and lack of material for re-analyses. However the sample at around 50 mg/mL indicated that this samples was also one of the most stable/less aggregating samples.
Aggregation of anti IL22R in F11-F14 was less pronounced compared to all other formulations, these formulations contained sucrose.

HMWP % for formulationis at ~150 mg/ml

| | F1 | F2 | F3 | F4 | F5 | F6 | F7 | F8 | F9 | F10 | F11 | F12 | F13 | F14 | F15 | F16 |
|---|---|---|---|---|---|---|---|---|---|---|---|---|---|---|---|---|
| t = 0 | 2.0 | 1.8 | 1.5 | 1.9 | 1.5 | 1.5 | 1.6 | 1.7 | 2.0 | 2.0 | 1.9 | 1.9 | 1.9 | 1.8 | 1.8 | 1.8 |
| 5 × F/T | 1.8 | 1.7 | 1.7 | 1.7 | 1.6 | 1.6 | 1.6 | 1.8 | 1.8 | 1.8 | 1.9 | 1.9 | 1.9 | 1.8 | 1.8 | 1.7 |
| 3 days@50° C. | 7.2 | 10.7 | 14.5 | 15.1 | 18.4 | 20.3 | 20.9 | 10.4 | 5.5 | 5.2 | 5.0 | 5.0 | 4.8 | 5.0 | NA | 11.0 |

HMWP % for “low concentration samples”

| | F1 | F2 | F3 | F4 | F5 | F6 | F7 | F8 | F9 | F10 | F11 | F12 | F13 | F14 | F15 | F16 |
|---|---|---|---|---|---|---|---|---|---|---|---|---|---|---|---|---|
| t = 0 | 2.1 | 1.9 | 2.0 | 2.0 | 2.3 | 2.3 | 2.4 | 2.5 | 2.7 | 2.5 | 2.5 | 2.5 | 2.6 | 2.5 | 2.5 | 2.2 |
| 5 × F/T | 2.1 | 2.1 | 2.2 | 2.2 | 2.1 | 2.1 | 2.2 | 2.3 | 2.8 | 2.5 | 2.3 | 2.3 | 2.3 | 2.2 | 2.2 | 3.2 |
| 3 days@50° C. | 1.4 | 1.4 | 1.6 | 3.7 | 6.1 | 8.1 | 8.2 | 3.6 | 1.3 | 1.9 | 1.6 | 1.5 | 1.7 | 2.6 | 1.6 | 3.2 |

### Example 11, Excipients and pH Effects on Viscosity

The influence of excipient combinations and anti IL22R concentration on viscosity was evaluated for five different anti-IL22R samples. Anti IL22R at 60 mg/mL in 20 mM histidine buffer pH 6.5 was buffer exchanged on MiniTrap DP 10 Columns from GE Helthcare into the respective formulations. After buffer exchange, the samples were concentrated to ˜150 mg/mL on amicon

Ultra-4 mL-MWCO 30K according to procedure B described in example 10. The excipient composition of the investigated formulations: F1, F7, F10, F11 and F12 is shown in formulation table in example 10 (using same formulation numbers).

Viscosity was measured by RheoSence microVisc viscometer at room temperature 18-22° C., shear rate of 1400 s⁻¹. The table below shows viscosity for samples at different protein concentrations.

| | F1 | | | F7 | | | | F10 | | F11 | | | | F12 | | | |
|---|---|---|---|---|---|---|---|---|---|---|---|---|---|---|---|---|---|
| Concentration (mg/mL) | 123 | 131 | 139 | 108 | 126 | 136 | 145 | 107 | 134 | 109 | 127 | 136 | 146 | 110 | 129 | 139 | 148 |
| viscosity (cP) | 5.1 | 5.6 | 6.8 | 3.2 | 4.2 | 5.0 | 5.3 | 3.7 | 6.5 | 3.7 | 5.1 | 6.3 | 7.2 | 4.8 | 6.7 | 7.6 | 8.9 |

All formulations contained 20 mM histidine, pH 6.0, and 0.2 mg/ml tween 20 (same as 0.02% w/w polysorbate 20. The table below outlines the formulation differences between F1, F7, F10, F11 and F12.

The data (table above) shows that the lowest viscosity is observed in F7 (this sample contains no disaccharide but a combination of four different amino acids: arginine, proline, glycine, methionine). F1 with 180 mM sucrose and 50 mM arginine has significantly higher viscosity at 139 mg/ml anti IL22R compared to F7 (the sucrose free sample) with about the same concentration (136 mg/mL), Both of these contain arginine. The highest viscosity is however found for F12 with proline, methionine, and sucrose. The viscosity of F10 (with 80 mM proline) was slightly higher than the viscosity of F11 (with no proline, but increased sucrose) when comparing samples of similar protein concentrations (134 mg/mL and 136 mg/mL respectively). Hence this suggest that proline increases the viscosity of anti IL22R. None of these contain arginine, and both contain 80 mM glycine.

| | F1 | F7 | F10 | F11 | F12 |
|---|---|---|---|---|---|
| Arg (mM) | 50 | 55 | | | |
| Pro (mM) | | 55 | 80 | | 80 |
| Gly (mM) | | 55 | 80 | 80 | |
| Met (mM) | | 40 | 40 | 40 | 40 |
| Sucrose (mM) | 180 | | 80 | 180 | 180 |

### Example 12, Excipient and pH Effects on Anti IL22R Stability

The influence of excipient combinations on biophysical properties of anti IL22R at ˜150 mg/ml was evaluated by comparison of 12 different formulations. 12 placebo formulations (formulations without anti IL22R and without Tween 20) were prepared and pH adjusted to 6.0. The anti IL22R formulations were prepared using an automated buffer exchange and concentration system, GRUNT from Unchained Labs. About 7.5 mL anti IL22R at 109 mg/mL was used for each formulation. This was buffer exchanges and concentrated into about 5 mL of final formulation. After preparation of these formulations (prior to addition of Tween 20) the formulations were sterile filtered. Hereafter Tween 20 stock solution was added to reach 0.2 mg/ml in final formulations, and pH and protein concentration was measured. The composition of the 12 formulations is outlined in the table below.

| | F1 | F11_a | F11_b | F11_c | F11_d | F11_e | F11_f | F11_g | F11_h | F11_i | F11_j | F11_k |
|---|---|---|---|---|---|---|---|---|---|---|---|---|
| Histidine | 20 | 20 | 20 | 20 | 20 | 20 | 20 | 20 | 20 | 20 | 20 | 20 |
| (mM) | | | | | | | | | | | | |
| Arginine | 50 | | | | | | | | | 50 | | |
| (mM) | | | | | | | | | | | | |
| Proline (mM) | | | | | | | | 80 | 80 | | | |
| Glycine | | 80 | 80 | 80 | 80 | 80 | 80 | 80 | | 60 | 120 | 180 |
| (mM) | | | | | | | | | | | | |
| Methionine | | 40 | 20 | 0 | 20 | 20 | 20 | 20 | 20 | 20 | 20 | 20 |
| (mM) | | | | | | | | | | | | |
| Sucrose | 180 | 180 | 180 | 180 | 180 | 180 | 180 | 100 | 180 | 100 | 140 | 80 |
| (mM) | | | | | | | | | | | | |
| Tween 20 | 0.2 | 0.2 | 0.2 | 0.2 | 0.2 | 0.2 | 0.2 | 0.2 | 0.2 | 0.2 | 0.2 | |
| (mg/mL) | | | | | | | | | | | | |
| pH | 6.08 | 6.07 | 6.09 | 6.05 | 5.43 | 5.71 | 6.6 | 6.05 | 6.09 | 6.09 | 6.06 | 6.09 |
| anti IL22R | 150.2 | 148.2 | 153.3 | 150.6 | 156.2 | 147.4 | 153.9 | 154.5 | 154 | 155.1 | 147.6 | 151.3 |
| (mg/mL) | | | | | | | | | | | | |

Analyses: The denaturation temperature of anti-IL22R in the different formulations was analysed by intrinsic fluorescence detection during heating scans using Uncle from Unchained Labs. The denaturation temperature (Td) was determined from the inflection point in the fluorescence vs temperature curve. Viscosity of the anti IL22R formulations shown in table above was measured by RheoSence microVisc viscometer (room temperature 18-22° C., shear rate of 1400 s⁻¹). 0.2 mg/ml tween 20 is the same as 0.02% (w/w) polysorbate 20.

Results and observations: The viscosity of formulations F1 and F11_i which contain 50 mM arginine was lower compared to all other formulations, see data table below. The highest viscosity was observed for F11_d, F 11_f and F11_h. Comparison of F11_b, F11_d, F11_e, F11_f suggests that optimal pH with regards to viscosity is in the range of 5.7-6.1. Comparison of F11_b and F11_h suggests that substituting proline with glycine decreases viscosity of anti IL22R formulations.

Denaturation temperatures (T_{d}) suggest that for the pH range 5.4-6.6 thermal stability of anti II22R increases with increasing pH. The thermal denaturation study also shows that arginine has a negative effect on the thermal stability of anti IL22R (relative low Td for F1 and F11_i). This is in accordance with data presented in example 6 and 10. Hence while having a positive influence on viscosity (reduces the viscosity of anti IL22R) arginine has a destabilizing impact as well. Comparison of T_{d} values for F11_a, F11_b and F11_c suggest that methionine up to 40 mM has only minor impact on the thermal stability of anti IL22. Comparison of T_{d} values for F1 and F11_i suggest that sucrose is a better stabilizer than glycine.

| | F1 | | F11_a | | F11_b | | F11_c | | F11_d | | F11_e | | F11_f | | F11_g | | F11_h | | F11_i | | F11_j | | F11_k | |
|---|---|---|---|---|---|---|---|---|---|---|---|---|---|---|---|---|---|---|---|---|---|---|---|---|
| Vis- | 8.6 | cP | 11.0 | cP | 11.6 | cP | 11.1 | cP | 12.4 | cP | 11.5 | cP | 12.4 | cP | 11.6 | cP | 12.4 | cP | 8.7 | cP | 11.0 | cP | 10.4 | cP |
| cosity | | | | | | | | | | | | | | | | | | | | | | | | |
| T_{d} | 65.1° | C. | 68° | C. | 67.6° | C. | 67.5° | C. | 64.8° | C. | 66.4° | C. | 68.1° | C. | 68.8° | C. | 66.8° | C. | 63.8° | C. | 66.6° | C. | 66.4° | C. |

Additional observations: During sterile filtration it was observed that anti IL22R formulations F11_d and F11_h were very difficult to filtrate. And more importantly sample coloration was observed by visual inspection for most of the formulations after 8 weeks storage at 40° C. Detailed route cause investigation was not introduced, but it was confirmed by MS peptide mapping (data not included) that sucrose was degraded and that anti IL22R was glycated

Since the glycoside bond in trehalose is more stable compared to the one in sucrose it was decided to test substitution of sucrose as disaccharide stabilizer with trehalose as disaccharide stabilizer. Trehalose was previous observed to increase the thermal stability of anti IL22R (example 5).

### Example 13: Substituting Sucrose with Trehalose, a More Stable Disaccharide

The influence of excipient combinations on biophysical properties of anti IL22R at ˜150 mg/mL was evaluated by comparison of 12 different formulations. 12 placebo formulations (formulations without anti IL22R and without Tween 20) were prepared and pH adjusted to 6.0. The anti IL22R formulations were prepared using an automated buffer exchange and concentration system, GRUNT from Unchained Labs. After preparation of these formulations (prior to addition of Tween 20) the formulations were sterile filtered. Hereafter Tween 20 stock solution was added to reach 0.2 mg/ml (same as 0.02% (w/w) polysorbate 20) in final formulations, and pH and protein concentration was measured. The composition of the 12 formulations is outlined in the table below.

| | F1 | F11_b | F11_e | F11_i | F11_k | F11_p | F11_r | F11_s | F11_t | F11_u | F11_v |
|---|---|---|---|---|---|---|---|---|---|---|---|
| His (mM) | 20 | 20 | 20 | 20 | 20 | 20 | 20 | 20 | 20 | 20 | 20 |
| Arg (mM) | 50 | | | 50 | | 50 | | 50 | 50 | | 50 |
| Asp (mM) | | | | | | 50 | | | 50 | | |
| Gly (mM) | | 80 | 80 | 60 | 180 | | 80 | 60 | | 180 | 160 |
| Met (mM) | | 20 | 20 | 20 | 20 | 20 | 20 | 20 | 20 | 20 | 20 |
| Suc (mM) | 180 | 180 | 180 | 100 | 80 | 100 | | | | | |
| Tre (mM) | | | | | | | 180 | 100 | 100 | 80 | |
| Tw20 | 0.2 | 0.2 | 0.2 | 0.2 | 0.2 | 0.2 | 0.2 | 0.2 | 0.2 | 0.2 | 0.2 |
| (mg/mL) | | | | | | | | | | | |
| pH | 6.0 | 6.1 | 5.7 | 6.0 | 6.0 | 6.0 | 6.0 | 6.0 | 6.0 | 6.0 | 6.0 |
| Conc. | 149.5 | 147.0 | 148.3 | 152.2 | 153.3 | 152.1 | 150.4 | 157.1 | 156.0 | 147.5 | 151.7 |
| (mg/mL) | | | | | | | | | | | |

Analyses: Viscosity of the anti IL22R formulations was measured by RheoSence microVisc viscometer (room temperature 18-22° C., shear rate of 1400 s⁻¹). SEC analyses was conducted using a TSKgel® SuperSW mAb HTP HPLC column. Mobile phase: 100 mM Sodium Phosphate, 300 mM Sodium Chloride, pH 6.8. Injection volume 0.1 uL. UV 280 nm detection. Undiluted samples were injected and analyzed. SEC integration procedure: HMWP % (total area percent of peaks eluting before the monomer peak.

| | F1 | F11_b | F11_e | F11_i | F11_k | F11_p | F11_r | F11_s | F11_t | F11_u | F11_v |
|---|---|---|---|---|---|---|---|---|---|---|---|
| His (mM) | 20 | 20 | 20 | 20 | 20 | 20 | 20 | 20 | 20 | 20 | 20 |
| Arg (mM) | 50 | | | 50 | | 50 | | 50 | 50 | | 50 |
| Asp (mM) | | | | | | 50 | | | 50 | | |
| Gly (mM) | | 80 | 80 | 60 | 180 | | 80 | 60 | | 180 | 160 |
| Met (mM) | | 20 | 20 | 20 | 20 | 20 | 20 | 20 | 20 | 20 | 20 |
| Suc (mM) | 180 | 180 | 180 | 100 | 80 | 100 | | | | | |
| Tre (mM) | | | | | | | 180 | 100 | 100 | 80 | |
| Tween 20 | 0.2 | 0.2 | 0.2 | 0.2 | 0.2 | 0.2 | 0.2 | 0.2 | 0.2 | 0.2 | 0.2 |
| (mg/mL) | | | | | | | | | | | |
| pH | 6.0 | 6.1 | 5.7 | 6.0 | 6.0 | 6.0 | 6.0 | 6.0 | 6.0 | 6.0 | 6.0 |
| Conc. | 149.5 | 147.0 | 148.3 | 152.2 | 153.3 | 152.1 | 150.4 | 157.1 | 156.0 | 147.5 | 151.7 |
| (mg/mL) | | | | | | | | | | | |

Results: Data shows that anti IL22R formulations at ˜150 mg/mL which contain 50 mM arginine have a viscosity, at room temperature, in the range of 7.2-7.9 cP whereas formulations without arginine have a measured viscosity which falls into the range of 9.7-11.1 cP. See data in table below.

| | F1 | F11_b | F11_e | F11_i | F11_k | F11_p | F11_r | F11_s | F11_t | F11_u | F11_v |
|---|---|---|---|---|---|---|---|---|---|---|---|
| Concentration | 149.5 | 147.0 | 148.3 | 152.2 | 153.3 | 152.1 | 150.4 | 157.1 | 156.0 | 147.5 | 151.7 |
| (mg/mL) | | | | | | | | | | | |
| Viscosity | 7.88 | 9.73 | 10.53 | 7.36 | 10.47 | 7.67 | 11.12 | 7.70 | 7.94 | 10.11 | 7.17 |
| (cP) | | | | | | | | | | | |

SEC data on the other hand shows that anti IL22R in formulations which contain 50 mM arginine has increased levels of protein aggregates (HMWP %) after 4 and 8 weeks at 40° C. (SEC data presented in table below). Arginine has consistently been shown to destabilize anti IL22R at high temperature (example 6, 10 and 12). F11_r can be regarded as a thermo-stable 150 mg/ml anti IL22R formulation.

Interestingly, longterm data (9 months at 5° C. and 9 months at 25° C.) revealed that 50 mM arginine can be present without compromising the stability if the storage temperature is decreased. The data for 9M@5° C. samples actually shows that the formulations with 50 mM arginine (F1, F11_s and F11_t) have slightly lower aggregate content compared to formulations without arginine (F11_r and F11_u).

The data for samples stored 9M@25° C. supports this finding (lower HMWP % in the presence of arginine) for formulations containing trehalose as stabilizing disaccharide, whereas F1 which contains sucrose and arginine has a relatively high aggregate level.

Hence, the presented data suggests that anti IL22R viscosity is decreased by arginine, that anti IL22R stability at 40° C. is reduced by arginine (due to increased aggregation), surprisingly that anti IL22R stability at 5° C. is increased by arginine, and that anti IL22R stability at temperatures ≥25° C. can be improved by substituting sucrose with trehalose.

| HMWP % | F1 | F11_b | F11_e | F11_i | F11_k | F11_p | F11_r | F11_s | F11_t | F11_u | F11_v |
|---|---|---|---|---|---|---|---|---|---|---|---|
| 4 weeks | 1.2 | 1.3 | 1.3 | 1.2 | 1.3 | 1.2 | 1.3 | 1.2 | 1.1 | 1.2 | 1.2 |
| at −80° C. | | | | | | | | | | | |
| 8 weeks | 1.2 | 1.3 | 1.1 | 1.1 | 1.3 | 1.1 | 1.3 | 1.1 | 1.1 | 1.3 | 1.1 |
| at 5° C. | | | | | | | | | | | |
| 12 weeks | 1.3 | 1.4 | 1.2 | 1.2 | 1.4 | 1.2 | 1.4 | 1.2 | 1.1 | 1.3 | 1.2 |
| at 5° C. | | | | | | | | | | | |
| 9 months | 1.9 | NA | NA | NA | NA | NA | 2.2 | 1.8 | 1.8 | 2.2 | 1.9 |
| at 5° C. | | | | | | | | | | | |
| 4 weeks | 1.2 | 1.3 | 1.2 | 1.2 | 1.4 | 1.2 | 1.6 | 1.1 | 1.2 | 1.3 | 1.2 |
| at 25° C. | | | | | | | | | | | |
| 8 weeks | 1.5 | 1.6 | 1.4 | 1.3 | 1.6 | 1.3 | 1.5 | 1.3 | 1.3 | 1.4 | 1.4 |
| at 25° C. | | | | | | | | | | | |
| 12 weeks | 1.7 | 1.7 | 1.6 | 1.4 | 1.9 | 1.4 | 1.7 | 1.4 | 1.4 | 1.8 | 1.5 |
| at 25° C. | | | | | | | | | | | |
| 9 months | 4.4 | NA | NA | NA | NA | NA | 3.2 | 2.6 | 2.8 | 3.3 | 2.8 |
| at 25° C. | | | | | | | | | | | |
| 4 weeks | 12.1 | 5.7 | 9.8 | 11.1 | 6.1 | 12.9 | 4.3 | 8.3 | 8.7 | 5.2 | 11.9 |
| at 40° C. | | | | | | | | | | | |
| 8 weeks | 24.5 | 11.2 | 14.1 | 18.9 | 9.8 | 15.8 | 7.9 | 15.4 | 15.4 | 9.5 | 21.7 |
| at 40° C. | | | | | | | | | | | |

### Example 14: Viscosity of Thermo-Stable Formulation

Viscosity curves (viscosity vs protein concentration) were made for anti IL22R formulation F11_r. The formulation was made by buffer exchange and concentration using MiniTrap DP 10 Columns 5 from GE Healthcare and amicon Ultra-4 mL-MWCO 30K according to procedure B described in example 11. The excipient composition of the investigated formulation: F11_r is shown in formulation table in example 14 (using same formulation number).

Analyses: Viscosity at 22° C. by RheoSence microVisc viscometer at room temperature 18-22° C. In this study “automatic shear rate” by the “Rheosense mvisc” was used for all samples. The actual 10 shear rate used for individual samples (ranging from about 50 s⁻¹ to 2400 s⁻¹) is indicated in the table below.

| | | | | | | | | | |
|---|---|---|---|---|---|---|---|---|---|
| concentration | 128.7 | 135.6 | 157.3 | 162.2 | 174.1 | 183.1 | 194.6 | 206.9 | 216.9 |
| (mg/mL) | | | | | | | | | |
| Viscosity (cP) | 6.8 | 8.5 | 10.8 | 14.2 | 17.4 | 22.3 | 28.7 | 37.2 | 48.1 |
| at 22° C. | | | | | | | | | |
| Shear Rate, | 1996 | 1481 | 1255 | 952 | 759 | 670 | 511 | 461 | 318 |
| 1/s | | | | | | | | | |

### Example 15: Methionine

The influence of the anti-oxidant methionine on anti IL22R stability was evaluated by comparison of six formulations with various concentration of methionine in the range of 0-30 mM. Formulations for this study were also prepared by the use of an automated buffer exchange system GRUNT. Anti IL22R, 102 mg/mL in histidine buffer pH 6.0 was buffer exchanged and concentrated to generate the formulations outlined below. After buffer exchange and concentration, the formulations were sterile filtered, and Tween 20 stock solution was added to reach 0.2 mg/mL in final formulations (same as 0.02% (w/w) polysorbate 20). The target pH and protein concentration should be similar for all samples, around pH 6 and protein concentration in the range of 150-155 mg/mL, both pH and protein concentration was measured (values shown in the last rows of table below). Each formulation is distributed in pre-fillable syringes, fill volume 1 mL, filling performed in a LAF bench.

| | F11_{—} | F11_{—} | F11_{—} | F11_{—} | F11_{—} | F11_{—} |
|---|---|---|---|---|---|---|
| | rz | ra | rb | rc | r | rd |
| Histidine (mM) | 20 | 20 | 20 | 20 | 20 | 20 |
| Glycine (mM) | 80 | 80 | 80 | 80 | 80 | 80 |
| Methionine (mM) | 0 | 5 | 10 | 15 | 20 | 30 |
| Trehalose (mM) | 180 | 180 | 180 | 180 | 180 | 180 |
| Tween 20 | 0.2 | 0.2 | 0.2 | 0.2 | 0.2 | 0.2 |
| (mg/mL) | | | | | | |
| pH | 6.1 | 6.1 | 6.1 | 6.1 | 6.1 | 6.1 |
| Anti IL22R | 153.0 | 154.0 | 155.2 | 157.0 | 156.9 | 156.0 |

Analyses: Anti IL22R methionine oxidation oxidation was analysed by peptide mapping after tryptic digestion followed by reduction of Anti IL22R using reversed phase chromatography and data dependent MS/MS detection on Thermo Q-Exactive hydrid orbitrap mass spectometer. MS/MS data was used for verification of the methionine oxidation positions and quantitation of the level of Methionine oxidation was carried out on MS data using Protein metrics Byonic/Byologic software. Peptide mapping MS was conducted after 2 months at 25° C. and 40° C. as well as after 7 months at 25° C. SEC analyses was conducted to quantify potential effects of methionine on anti IL22R aggregation (HMWP %) the analyses was performed initially (tz) as well as after after 1 month and 2 months at 5° C., 25° C. and 40° C.

Results: Methionine oxidation was detected at four methionie residues in the Anti IL22R heavy chain (HC): M255 (most suseptable to oxidation), M34, M83 and M431 (second most suspetable to oxidation). The data is shown as total increase in met oxidation for all four positions in table below. The data clearly show that methionine added to the formulation can limit methionine residue oxidation. The MS data supports selection of methionine in formulation at a concentration >5 mM

| | F11_rz | F11_ra | F11_rb | F11_rc | F11_r | F11_rd |
|---|---|---|---|---|---|---|
| 2 M at 25° C. | 5.5 | 3.7 | 3.7 | 3.7 | 3.6 | 3.8 |
| 2 M at 40° C. | 11.6 | 6.4 | 5.2 | 4.7 | 4.7 | 4.3 |
| 7 M at 25° C. | 12.4 | 6.3 | 5.5 | 5.2 | 5.6 | 5.2 |

The SEC data suggests that methionine has a stabilizing effect in terms of preventing anti IL22R aggregation. For formulations exposed to 40° C. the lowest aggregate content (HMWP %) is observed for formulation containing 15 mM methionine (1 and 2 months data), whereas lowest HMWP levels after storage at 25° C. (2 months) is observed for formulations containing 20 mM and 30 mM methionine. HMWP data is presented in table below. No effects of methionine on fragmentation (LMWP %) was observed by the SEC analyses (one sample F11_rc however had unexpected high LMWP after 1 months at 5° C., this is regarded as an outlier and might be due to mistake in the sample set-up).

| | | | | | | |
|---|---|---|---|---|---|---|
| | F11_rz | F11_ra | F11_rb | F11_rc | F11_r | F11_rd |
| Methionine | 0 mM | 5 mM | 10 mM | 15 mM | 20 mM | 30 mM |

HMWP %

| | | | | | | |
|---|---|---|---|---|---|---|
| TZ | 1.12 | 1.09 | 1.09 | 1.11 | 1.13 | 1.08 |
| 1 M 5° C. | 1.26 | 1.28 | 1.27 | 1.24 | 1.26 | 1.24 |
| 1 M 25° C. | 1.66 | 1.57 | 1.58 | 1.62 | 1.54 | 1.53 |
| 1 M 40° C. | 6.44 | 5.66 | 5.70 | 4.90 | 4.96 | 5.17 |
| 2 M 5° C. | 1.43 | 1.38 | 1.39 | 1.38 | 1.32 | 1.36 |
| 2 M 25° C. | 2.07 | 1.96 | 1.89 | 1.85 | 1.76 | 1.76 |
| 2 M 40° C. | 12.11 | 9.79 | 9.26 | 8.92 | 9.07 | 9.45 |

LMWP %

| | | | | | | |
|---|---|---|---|---|---|---|
| TZ | 2.48 | 2.60 | 2.62 | 2.41 | 2.60 | 2.44 |
| 1 M 5° C. | 1.98 | 1.96 | 1.99 | 4.61 | 1.96 | 2.01 |
| 1 M 25° C. | 2.55 | 2.51 | 2.49 | 2.59 | 2.57 | 2.51 |
| 1 M 40° C. | 4.94 | 4.85 | 4.84 | 4.63 | 4.64 | 4.83 |
| 2 M 5° C. | 1.85 | 1.81 | 1.85 | 1.87 | 1.75 | 1.86 |
| 2 M 25° C. | 2.65 | 2.56 | 2.56 | 2.56 | 2.54 | 2.58 |
| 2 M 40° C. | 6.46 | 6.32 | 6.40 | 6.36 | 6.49 | 6.53 |

All in all, the data indicates that 10-30 mM methionine is beneficial for chemical and physical stabilization of Anti IL22R in terms of reducing protein oxidation and aggregation.

### Example 16: Tween 20 (Polysorbate 20)

There is a wish to add Tween 20 to anti IL22R formulations (and protein formulations in general) to limit surface adsorption. The influence of Tween 20 on anti IL22R aggregation and viscosity was evaluated. Anti IL22R at 139.5 mg/mL in 20 mM histidine pH 6.0 was buffer exchanged into a buffer containing: 20 mM histidine, 80 mM glycine, 20 mM methionine, 180 mM trehalose, pH 6.0 using MiniTrap DP 10 Columns from GE Healthcare and concentrated to 150 mg/mL on amicon Ultra-4 mL-MWCO 30K. After buffer exchange and up-concentration, the formulations were sterile filtered and divided in different aliquots to prepare formulations with various tween 20 concentrations. Tween 20 stock solution was added to reach: 0.2 mg/ml, 0.4 mg/mL and 0.8 mg/ml (same as 0.02% (w/w), 0.04% (w/w) and 0.08% (w/w) polysorbate 20) in final formulations, one formulation was kept tween 20 free. The compared formulations are outlined in table below. Osmolality, pH and protein concentration was measured (by freezing point osmometry using Osmomat 3000, Gonotec, by standard pH meter and by standard UV280 method on lunatic from unchained lab. SEC analyses and viscosity measurements were conducted as described in example 13.

| | F11_w | F11_r | F11_x | F11_y |
|---|---|---|---|---|
| Histidine (mM) | 20 | 20 | 20 | 20 |
| Glycine (mM) | 80 | 80 | 80 | 80 |
| Methionine (mM) | 20 | 20 | 20 | 20 |
| Trehalose (mM) | 180 | 180 | 180 | 180 |
| Tween 20 | | 0.2 | 0.4 | 0.8 |
| (mg/mL) | | | | |
| pH | 6.3 | 6.3 | 6.3 | 6.3 |
| Anti IL22R | 156.3 | 155.8 | 154.9 | 154.4 |
| (mg/mL) | | | | |
| Osmolality | 335 | 331 | 330 | 321 |
| (mOsm/kg) | | | | |

Results: The SEC analyses showed that Tween 20 had no influence in terms of preventing anti IL22R aggregation. The SEC quantified HMWP % for all samples in shown in table below.

| | F11_w | F11_r | F11_x | F11_y |
|---|---|---|---|---|
| | (HMWP %) | (HMWP %) | (HMWP %) | (HMWP %) |
| TZ | 1.7 | 1.8 | 1.7 | 1.7 |
| 4 w 25 C. | 1.6 | 1.6 | 1.6 | 1.6 |
| 4 w 40 C. | 5.3 | 5.8 | 5.7 | 5.8 |
| 8 w 5 C. | 1.6 | 1.6 | 1.6 | 1.6 |
| 8 w 25 C. | 1.9 | 2.0 | 2.0 | 2.0 |
| 8 w 40 C. | 6.9 | 7.1 | 7.1 | 7.1 |

Viscosity was measured for all four formulations, for F11_w, F11_r and F11_y the sample amount was sufficient to measure viscosity at different concentrations. No influence of tween 20 on viscosity.

| | conc. | viscosity | | conc. | Viscosity |
|---|---|---|---|---|---|
| Formul. | (mg/mL) | (cP) | Formul. | (mg/mL) | mPa-s |
| F11_w | 155.1 | 12.1 | F11_x | 154.9 | 11.5 |
| | 142.6 | 9.5 | | | |
| | 135.6 | 7.9 | | | |
| | 125.1 | 6.4 | | | |
| F11_r | 153.3 | 11.7 | F11_y | 153.8 | 11.6 |
| | 138.8 | 8.5 | | 140.5 | 8.9 |
| | 129.0 | 6.8 | | 131.8 | 7.3 |
| | 120.3 | 5.7 | | 118.9 | 5.6 |

### Example 17: Tween 20, Methionine, and pH Effect on Anti IL22R Formulations at 150 mg/ml

In this study 150 mg/mL anti IL22R formulations was prepared to study the influence of: +/−0.2 mg/mL Tween 20, +/−20 mM methionine, pH 6.0 vs. 6.5 and omitted trehalose/glycine concentration, on the long-term stability of anti IL22R at ±5° C. and ±25° C. as well as on shorter term stability at 40° C. The composition of the formulations is shown in table below. 102 mg/ml anti IL22R in 20 mM histidine pH 6.0 was used as starting material for the preparation of different formulations. The protein was diluted to 50 mg/mL with the respective diafiltration buffers (tween free placebo formulations) prior diafiltration. Following diafiltration a final concentration step up to around 175-200 mg/mL was performed prior product displacement with diafiltration buffer (the target concentration for final anti IL22R formulation was around 150 mg/mL). After diafiltration and concentration the formulations were sterile filtered, and Tween 20 stock solution was added to reach 0.2 mg/ml (same as 0.02% (w/w) polysorbate 20) in final formulations. Osmolality, pH and protein concentration was measured (as described in example 16). The formulations were filled in PFS and stored at different temperatures.

| | F11_r | F11_rx | F11_ry | F11_rz | F11_u |
|---|---|---|---|---|---|
| Histidine (mM) | 20 | 20 | 20 | 20 | 20 |
| Glycine (mM) | 80 | 80 | 80 | 80 | 180 |
| Methionine (mM) | 20 | 20 | 20 | 0 | 20 |
| Trehalose (mM) | 180 | 180 | 180 | 180 | 80 |
| Tween20 (mg/mL) | 0.2 | 0 | 0.2 | 0.2 | 0.2 |
| Osmolality (mOsm/kg) | 373 | 372 | 364 | 346 | 346 |
| pH | 6.04 | 5.99 | 6.53 | 5.97 | 6.02 |
| anti IL22R concentration | 147 | 143 | 152 | 155 | 152 |
| (mg/mL) | | | | | |
| Viscosity at 22° C. (cP) | 11.3 | 10.3 | 12.7 | 13.6 | 11.7 |

Analyses: SEC analyses was conducted using a Agilent AdvanceBio SEC 200 Å, 1.9 μm, 4.6 mm×150 mm column. Mobile phase: 100 mM Sodium Phosphate, 300 mM Sodium Chloride, pH 6.8. Injection volume 0.1 uL. Undiluted samples were injected and analyzed. UV 280 nm detection. SEC integration procedure: HMWP % (total area percent of peaks eluting before the monomer peak. CIEX analyses was conducted using MabPac SCX-10 RS 50×2.1 mm, 5 μm. Column temperature 25° C. Mobile phase A: Diluted CX-1 pH gradient buffer A pH 5.6, from Thermo Scientific (10× diluted in milliQ water). Mobile phase B: Diluted CX-1 pH gradient buffer B pH 10.2, from Thermo Scientific (10× diluted in milliQ water). Flow rate: 0.4 mL/min Detection 280 nm and 215 nm. Run time 23 minutes. Gradient: (Initial: 10% B held at 2 min. Sample separation: Linear gradient 10-40% B over 13 min. Change mobile phase to 90% B hold for 3 minutes. Returned to the starting solvent 10% B hold for 5 min. CIEX integration procedure: Acidic peaks % (total area percent of peaks eluting before the main charge variant), data shown in table below. Basic peaks % (total area percent of peaks eluting after the main charge variant), data not shown in table below, since not relevant for the conclusion.

Results: The CIEX analyses indicates that chemical stability is higher at pH 6.0 compared to pH 6.5. This is observed as larger increase in acidic peaks (compared to t=0, tz values) at most sampling points for formulation F11_ry (CIEX data is shown in the upper table of the two tables below). Increase in acidic peak area was found by MS characterization to correlate with increased deamidation. The SEC data shows that also the colloidal stability was found to be higher at pH 6.0 compared to pH 6.5 (SEC data is shown in the lower table of the two tables below). Comparison of F11_r and F11_ry shows that HMWP % is almost consistently higher at pH 6.5, for a few sampling points the formulation at pH 6.5 also appear to have higher amounts of anti IL22R fragments, LMWP %.

Supplement information that could not be used for ranking between the different formulations, but supports the buffer selection (20 mM histidine): The measured pH at all time points was stable over 2 years.

| | | | | | | | | | | |
|---|---|---|---|---|---|---|---|---|---|---|
| CIEX | | 1 M@25 | 1 M@40 | 3 M@5 | 3 M@25 | 3 M@40 | 6 M@5 | 6 M@25 | 23 M −80 | 23 M 5° |
| data | TZ | C. | C. | C. | C. | C. | C. | C. | ° C. | C. |

Acidic Peaks (area %)

| | | | | | | | | | | |
|---|---|---|---|---|---|---|---|---|---|---|
| F11_r | 13.0 | 14.3 | 25.3 | 13.4 | 17.6 | 46.1 | 13.6 | 23.3 | 12.6 | 15.5 |
| F11_rx | 13.0 | 14.3 | 25.1 | 13.3 | 17.2 | 45.7 | 14.2 | 23.6 | 12.9 | 15.3 |
| F11_ry | 13.3 | 15.7 | 31.1 | 14.0 | 21.0 | 57.6 | 15.3 | 30.5 | 13.3 | 18.4 |
| F11_rz | 13.3 | 14.5 | 25.2 | 13.5 | 17.6 | 45.0 | 14.5 | 24.1 | 13.1 | 15.5 |
| F11_u | 13.0 | 14.4 | 26.3 | 13.3 | 18.0 | 48.0 | 14.4 | 25.5 | 12.8 | 15.9 |

Main Charge variant (area %)

| | | | | | | | | | | |
|---|---|---|---|---|---|---|---|---|---|---|
| F11_r | 51.4 | 51.3 | 41.8 | 51.5 | 51.7 | 27.4 | 54.5 | 52.2 | 48.3 | 50.2 |
| F11_rx | 51.4 | 51.9 | 41.8 | 51.7 | 51.2 | 27.3 | 53.9 | 50.6 | 48.5 | 49.6 |
| F11_ry | 51.3 | 51.2 | 39.7 | 51.4 | 49.4 | 22.6 | 53.2 | 46.2 | 48.7 | 48.6 |
| F11_rz | 51.3 | 51.2 | 41.3 | 51.5 | 50.4 | 25.7 | 53.5 | 49.6 | 48.5 | 49.2 |
| F11_u | 51.6 | 51.8 | 41.0 | 52.2 | 50.7 | 26.0 | 53.7 | 49.5 | 48.5 | 49.4 |

| | | | | | | | | | | | |
|---|---|---|---|---|---|---|---|---|---|---|---|
| SEC | | 1 M | 1 M | 3 M | 3 M | 3 M | 6 M | 6 M | 23 M −80° | 23 M | 23 M |
| data | TZ | 25° C. | 40° C. | 5° C. | 25° C. | 40° C. | 5° C. | 25° C. | C. | 5° C. | 25° C. |

HMWP (area %)

| | | | | | | | | | | | |
|---|---|---|---|---|---|---|---|---|---|---|---|
| F11_r | 1.3 | 1.5 | 3.8 | 1.5 | 1.8 | 7.6 | 1.7 | 2.5 | 1.2 | 2.2 | 4.5 |
| F11_rx | 1.2 | 1.4 | 3.7 | 1.7 | 1.7 | 6.7 | 1.6 | 2.3 | 1.2 | 2.0 | 3.8 |
| F11_ry | 1.6 | 1.9 | 4.2 | 1.4 | 2.6 | 6.2 | 2.2 | 3.4 | 1.4 | 3.0 | 5.7 |
| F11_rz | 1.3 | 1.6 | 4.9 | 1.4 | 2.1 | 11.6 | 1.8 | 3.2 | 1.2 | 2.5 | 6.6 |
| F11_u | 1.2 | 1.5 | 4.2 | 1.4 | 2.0 | 9.3 | 1.7 | 2.7 | 1.2 | 2.2 | 4.9 |

Monomer (area %)

| | | | | | | | | | | | |
|---|---|---|---|---|---|---|---|---|---|---|---|
| F11_r | 97.1 | 96.6 | 93.2 | 97.0 | 96.2 | 86.6 | 96.4 | 94.5 | 97.1 | 96.7 | 90.7 |
| F11_rx | 97.1 | 96.8 | 93.0 | 96.8 | 96.4 | 87.2 | 96.4 | 94.7 | 97.1 | 96.8 | 91.7 |
| F11_ry | 96.8 | 96.4 | 92.3 | 97.0 | 95.3 | 87.4 | 95.8 | 93.4 | 96.8 | 95.8 | 89.2 |
| F11_rz | 97.2 | 96.8 | 91.8 | 97.1 | 95.9 | 82.7 | 96.2 | 93.6 | 97.2 | 96.5 | 88.8 |
| F11_u | 97.2 | 96.7 | 92.9 | 97.0 | 95.9 | 84.9 | 96.3 | 94.1 | 97.2 | 96.7 | 90.4 |

LMWP (area %)

| | | | | | | | | | | | |
|---|---|---|---|---|---|---|---|---|---|---|---|
| F11_r | 1.6 | 1.8 | 3.0 | 1.5 | 2.0 | 5.9 | 2.0 | 3.1 | 0.9 | 1.1 | 4.8 |
| F11_rx | 1.6 | 1.8 | 3.3 | 1.5 | 2.0 | 6.1 | 2.0 | 3.1 | 0.8 | 1.2 | 4.5 |
| F11_ry | 1.7 | 1.7 | 3.6 | 1.6 | 2.1 | 6.5 | 2.0 | 3.2 | 0.8 | 1.1 | 5.1 |
| F11_rz | 1.5 | 1.6 | 3.3 | 1.5 | 2.0 | 5.7 | 2.0 | 3.2 | 0.8 | 1.1 | 4.6 |
| F11_u | 1.6 | 1.7 | 2.9 | 1.5 | 2.0 | 5.9 | 2.0 | 3.1 | 0.8 | 1.1 | 4.7 |

### Example 18: Arginine Effects on Viscosity at about 100-200 mg/mL Anti IL22R and Stability

In this study highly concentrated anti IL22R formulations with different arginine concentration was prepared to study the influence of arginine on viscosity and stability at ±5° C. and ±25° C. It was decided to maintain osmolality at similar levels between formulations (to maintain a relevant osmolality level for sc injection), which means that the concentration of trehalose and/or glycine was decreased concomitant to increased arginine concentrations. The composition of the buffers are shown in table below (note that the F11_s1 placebo formulations in this example has similar composition as F11_s in example 13). 102 mg/ml anti IL22R in 20 mM histidine pH 6.0 was used as starting material for the preparation of different formulations. The protein (85 mL at 102 mg/mL) was diluted to 50 mg/mL with the diafiltration buffer (tween free placebo formulations) prior diafiltration. Following diafiltration a final concentration step to around 200-220 mg/ml was performed prior product displacement with diafiltration buffer. After diafiltration and concentration the formulations were sterile filtered, and Tween 20 stock solution was added to reach 0.2 mg/ml in final formulations (same as 0.02% (w/w) polysorbate 20). Osmolality (by freezing point osmometry on Osmomat 3000, Gonotec), pH and protein concentration (by UV absorption on nano drop, Thermo Scientific) was measured.

| | F11_s1 | F11_s2 | F11_s3 | F11 s4 |
|---|---|---|---|---|
| Histidine (mM) | 20 | 20 | 20 | 20 |
| Arginine (mM) | 50 | 50 | 80 | 100 |
| Glycine (mM) | 60 | | | |
| Methionine (mM) | 20 | 20 | 20 | 20 |
| Trehalose (mM) | 100 | 140 | 100 | 60 |
| Tween20 (mg/mL) | 0.2 | 0.2 | 0.2 | 0.2 |
| Osmolality, placebo | 314 | 294 | 303 | 294 |
| (mOsm/kg) | | | | |
| Osmolality (mOsm/kg) | 356 | 346 | 358 | 335 |
| pH | 6.0 | 6.0 | 6.0 | 6.0 |
| anti IL22R concentration | 196 | 200 | 212 | 225 |
| (mg/mL) * | | | | |

* 1:1 dilution of samples was made prior to concentration measurements

Analyses: The viscosity measurements of anti IL22R at different concentrations in formulation F11_s1, F11_s2, F11_s3, F11_s4 was performed using RheoSence microVisc viscometer (22° C., shear rate of 1400 s⁻¹). Anti IL22R concentration series for viscosity measurements in the range of about 100-200 mg/mL were made by diluting the protein formulations with respective placebo formulations. Viscosity of high concentrated anti IL22R in F11_s2, F11_s3, F11_s4 was furthermore determined by Dynapro DLS plate reader III from Wyatt to analyze effect of temperature on the viscosity. Viscosity data is shown in tables below. Stability was evaluated by a variety of stability indicating methods. In this example CIEX and SEC data is shown since these analyses shows some stability differences during 9 months storage. The SEC and CIEX analysis was performed as described in example 17.

Results: The microVisc based viscosity data for anti IL22R at different concentrations in formulation F11_s1, _s2, _s3 and _s4 is shown in table below. Exponential fit of the viscosity vs. concentration data shows that anti IL22R has similar viscosity profile in F11_s1 and F11_s2, this suggests that in the presence of 50 mM arginine, glycine have no viscosity reducing influence on anti IL22R which was indicated for arginine free formulations (example 12 and 13).

The present data also shows that increasing the arginine concentration from 50 mM (F11_s1 and F11_s2) to 80-100 mM (F11_s3 and F11_s4) reduces the viscosity of anti IL22R.

| | | Visc. |
|---|---|---|
| | Conc. | (cP) |
| Formulation | mg/mL | 22° C. |
| F11_s1 | 197.5 | 19.4 |
| | 191.8 | 17.1 |
| | 179.3 | 12.8 |
| | 173.3 | 12.0 |
| | 158.1 | 8.1 |
| | 147.1 | 7.2 |
| | 134.8 | 5.5 |
| | 107.0 | 3.4 |
| F11_s2 | 196.0 | 19.3 |
| | 183.9 | 15.0 |
| | 167.0 | 11.0 |
| | 174.9 | 11.7 |
| | 155.5 | 8.9 |
| | 152.7 | 7.7 |
| | 131.5 | 5.1 |
| | 105.9 | 3.4 |
| F11_s3 | 205.3 | 19.5 |
| | 187.8 | 13.3 |
| | 179.3 | 11.8 |
| | 173.4 | 10.6 |
| | 158.0 | 7.4 |
| | 143.6 | 6.3 |
| | 134.1 | 5.1 |
| | 113.7 | 3.6 |
| F11_s4 | 217.4 | 22.0 |
| | 208.0 | 16.5 |
| | 190.2 | 13.3 |
| | 182.0 | 12.0 |
| | 167.3 | 7.9 |
| | 154.7 | 7.0 |
| | 138.0 | 5.5 |
| | 118.6 | 3.8 |

*concentrations were measured undiluted, an equipment warning was made for the sample since the concentration was too high for proper measurement (the concentration might be higher than indicated by measurement)

The influence of arginine on the viscosity of anti IL22R is even more clear when the present data for highly concentrated anti IL22R in F11_s3 and F11_s4 is compared to the viscosity data presented in example 14 for anti IL22R in F11_r (without arginine) where the viscosity increases from about 29 to 48 cP in the concentration range 195-217 mg/mL.

The viscosity was furthermore evaluated for highly concentrated formulations at different temperatures. Viscosity increases with decreasing temperature, and the viscosity reducing effect of arginine on anti IL22R formulations appears more pronounced at lower temperatures. From this data the following effects on viscosity appears clear:
Protein concentration in the range of 200-212 mg/mL has significant influence on viscosity (see data for F11_s3). Hence only pairwise comparison between 200 mg/mL formulations and 212/213 mg/mL formulations is relevant.
Arginine decreases viscosity (F11_s2 contains 50 mM arginine, F11_s3 80 mM arginine and F11_s4 100 mM arginine)

| Viscosity | | | | |
|---|---|---|---|---|
| data (cP) at | F11_s2 | F11_s3 | F11_s3 | F11_s4 |
| different | 200 | 200 | 212 | 213 |
| temperatures | mg/mL | mg/mL | mg/mL | mg/mL |
| 25° C. | 13.8 | 13.5 | 16.3 | 16.0 |
| 20° C. | 20.4 | 19.8 | 24 | 23.9 |
| 15° C. | 28.1 | 26.5 | 34.1 | 29.4 |
| 10° C. | 42.8 | 34.7 | 44.6 | 42.3 |

Stability results: Very similar stability profiles were observed for aniti IL22R in F11_s1, F11_2, F11_3 and F11_4, see SEC data in table below. However, after 9 months storage at 25° C. the analyses reveal some differences (last/right column in table below). Comparison of F11_s3 at 212 mg/mL and F11_s4 at 214 mg/mL, suggests F11_s3 to be slightly more stabilizing in terms of reducing anti IL22R aggregation (these samples can be compared since they are similar in protein concentration). Lower anti IL22R aggregation can be caused by the higher concentration of trehalose, 100 mM, in F11_s3 compared 60 mM in F11_s4 (in line with data from example 5 and 13 showing stabilizing effects of trehalose) and/or by decreasing the arginine concentration from 100 to 80 mM. Comparison of F11_s2 and F11_s3 at 200 mg/mL indicates that increasing trehalose from 100 mM to 140 mM and concomitantly reduce arginine from 80 mM to 50 mM only have very small influence on HMWP % after 9M at 25° C. Hence the stability of anti IL22R seems comparable in F11_2 and F11_3) whereas the viscosity data showed benefit of formulating the mAb in F11_s3 (compared to F11_s2).

As expected, small increases in protein concentration are associated with small increases in aggregation. However, for these stable formulations, very small differences are observed when comparing the HMWP values for F11_s3 at 200 mg/mL and at 212 mg/mL as well as HMWP values for F11_s4 at 213 mg/ml and at 217-225 mg/mL.

| | | | | | | | | |
|---|---|---|---|---|---|---|---|---|
| | 1 M | 3 M | 3 M | 5 M | 5 M | TZ | 9 M | 9 M |
| T0 | 25° C. | 5° C. | 25° C. | 5° C. | 25° C. | 9 M | 5° C. | 25° C. |

HMWP (area %)

| | | | | | | | | | |
|---|---|---|---|---|---|---|---|---|---|
| F11_s1 196 mg/mL | 1.2 | 1.6 | 1.4 | 1.9 | 1.5 | 2.3 | 1.2 | 1.5 | 2.7 |
| F11_s2 200 mg/mL | 1.2 | 1.7 | 1.4 | 2.0 | 1.5 | 2.3 | 1.2 | 1.6 | 2.7 |
| F11_s3 200 mg/mL | 1.2 | 1.5 | 1.3 | 2.0 | 1.4 | 2.3 | 1.2 | 1.4 | 2.8 |
| F11_s3 212 mg/mL | 1.2 | 1.7 | 1.4 | 2.1 | 1.4 | 2.4 | 1.2 | 1.5 | 2.9 |
| F11_s4 213 mg/mL | 1.2 | 1.6 | 1.4 | 2.0 | 1.4 | 2.5 | 1.2 | 1.4 | 3.2 |
| F11_s4 217-225 | 1.2 | 1.8 | 1.4 | 2.2 | 1.5 | 2.6 | 1.2 | 1.5 | 3.3 |
| mg/mL* | | | | | | | | | |

Monomer (area %)

| | | | | | | | | | |
|---|---|---|---|---|---|---|---|---|---|
| F11_s1 196 mg/mL | 98.2 | 97.6 | 98.0 | 96.9 | 97.8 | 96.2 | 97.9 | 97.6 | 94.8 |
| F11_s2 200 mg/mL | 98.2 | 97.5 | 97.9 | 96.8 | 97.8 | 96.2 | 97.8 | 97.4 | 94.8 |
| F11_s3 200 mg/mL | 98.2 | 97.6 | 98.0 | 96.9 | 97.9 | 96.2 | 97.9 | 97.7 | 94.8 |
| F11_s3 212 mg/mL | 98.2 | 97.4 | 98.0 | 96.7 | 97.9 | 96.1 | 97.9 | 97.6 | 94.6 |
| F11_s4 213 mg/mL | 98.1 | 97.5 | 98.0 | 96.8 | 97.9 | 96.0 | 98.0 | 97.6 | 94.5 |
| F11_s4 217-225 | 98.1 | 97.4 | 97.9 | 96.6 | 97.8 | 95.8 | 98.0 | 97.5 | 94.4 |
| mg/mL* | | | | | | | | | |

LMWP (area %)

| | | | | | | | | | |
|---|---|---|---|---|---|---|---|---|---|
| F11_s1 196 mg/mL | 0.6 | 0.9 | 0.7 | 1.2 | 0.7 | 1.5 | 0.9 | 0.9 | 2.5 |
| F11_s2 200 mg/mL | 0.6 | 0.9 | 0.7 | 1.2 | 0.7 | 1.5 | 1.0 | 1.0 | 2.4 |
| F11_s3 200 mg/mL | 0.6 | 0.9 | 0.7 | 1.2 | 0.7 | 1.5 | 1.0 | 0.9 | 2.5 |
| F11_s3 212 mg/mL | 0.6 | 0.9 | 0.7 | 1.2 | 0.7 | 1.5 | 0.9 | 1.0 | 2.5 |
| F11_s4 213 mg/mL | 0.7 | 0.9 | 0.7 | 1.2 | 0.7 | 1.5 | 0.9 | 1.0 | 2.4 |
| F11_s4 217-225 | 0.7 | 0.9 | 0.7 | 1.2 | 0.7 | 1.5 | 0.8 | 1.0 | 2.3 |
| mg/mL* | | | | | | | | | |

*some variation in concentration determinations were observed

CIEX data also reveals very similar stability profiles between the formulations, with a small tendency of F11 s3 to be more stable than F11 s1 in terms of lower increase in acidic charge variants and better maintenance of main charge variant.

| | Acidic peaks (area %) | | | Main charge varian (area %) | | | Basic peaks (area %) | | |
|---|---|---|---|---|---|---|---|---|---|
| | 9 M −80° | 9 M | 9 M | 9 M −80° | 9 M | 9 M | 9 M −80° | 9 M | 9 M |
| CIEX data | C. | 5° C. | 25° C. | C. | 5° C. | 25° C. | C. | 5° C. | 25° C. |
| F11_s1 196 | 12.9 | 13.3 | 25.6 | 48.2 | 48.0 | 40.8 | 38.8 | 38.7 | 33.6 |
| mg/mL | | | | | | | | | |
| F11_s2 200 | 12.8 | 13.3 | 24.7 | 47.9 | 48.1 | 41.5 | 39.3 | 38.6 | 33.8 |
| mg/mL | | | | | | | | | |
| F11_s3 200 | 12.7 | 13.0 | 24.4 | 48.2 | 48.3 | 41.8 | 39.1 | 38.7 | 33.8 |
| mg/mL | | | | | | | | | |
| F11_s3 212 | 12.8 | 13.0 | 24.5 | 47.9 | 48.1 | 42.0 | 39.4 | 38.8 | 33.5 |
| mg/mL | | | | | | | | | |
| F11_s4 213 | 12.8 | 13.2 | 24.3 | 48.1 | 48.0 | 41.7 | 39.1 | 38.8 | 34.0 |
| mg/mL | | | | | | | | | |
| F11_s4 217-225 | 12.8 | 13.2 | 24.6 | 48.1 | 48.3 | 41.3 | 39.1 | 38.5 | 34.1 |
| mg/mL* | | | | | | | | | |

In summary, preparation/concentration data, viscosity data and stability data guide the excipient balance to achieve highly concentrated and stable anti IL22R at around 200-225 mg/mL.

### Example 19: Arginine Effects on Viscosity and Injectability

In this study viscosity and injectability were explored for different anti IL22R formulations at different anti IL22R concentrations. Injectability as well as flexibility in relation selection of syringes and needle sizes is believed to be affected by viscosity. In this study injectability tests were conducted for different anti IL22R formulations when injected through prefilled syringes, PFS BD NEOPAK 1 mL ½ inch 27G STW needle, or through PFS BD NEOPAK 1 mL ½ inch 29 TW needle. Injection force measurements is relevant for evaluation of the injectability of a pharmaceutical product. A regulatory guideline for manually injection suggests a maximum injection force of 25 Newton at ≥18° C., and as a rule of thumb, subcutaneous injections (independent of dose volume) should be performed within 10 seconds.

The anti IL22R formulations used for injectability testing were prepared 102 mg/mL anti IL22R in 20 mM histidine pH 6.0 as starting material. This material was buffer exchange (into the formulations shown in table below) and up-concentration was performed using PD-10 desalting columns Sephadex G-25 Medium from GE-Healthcare and Amicon Ultra-15 centrifugal filter unit MWCO 50 kDa from Millipore. The 4 formulations were sterile filtered through a 0.22 μm filter followed by Tween 20 addition.

Analyses: Viscosity was measured as in example 18. Injection force was measured using Instron 5564 with following test parameters: 1 mL injection volume, injection rate 1 mL within 5 sec.

| | F11_r | F11_s2 | F11_s3 | F11_s4 |
|---|---|---|---|---|
| Histidine | 20 | 20 | 20 | 20 |
| (mM) | | | | |
| Arginine | | 50 | 80 | 100 |
| (mM) | | | | |
| Glycine (mM) | 60 | | | |
| Methionine | 20 | 20 | 20 | 20 |
| (mM) | | | | |
| Trehalose | 180 | 140 | 100 | 60 |
| (mM) | | | | |
| Tween20 | 0.2 | 0.2 | 0.2 | 0.2 |
| (mg/mL) | | | | |
| pH | 6.0 | 6.0 | 6.0 | 6.0 |

The viscosity data show that the viscosity of F11_r has surpassed 20 cP at 181.3 mg/ml anti IL22R, for F11_s2 20 cP is surpassed for the 206.8 mg/ml sample, whereas F11_s3 and F11_s4 do not reach 20 cP even at the highest anti IL22R concentrations of 205.3 mg/ml and 213.4 mg/ml respectively. Data presented below shows all viscosity data for F11_r, F11_s2, F11_s3 and F11_s4 generated during exp. 18 and 19.

| F11_r, | F11_r | F11_s2, | F11_s2 | F11_s3, | F11_s3 | F11_s3, | F11_s3 |
|---|---|---|---|---|---|---|---|
| Conc. | Viscosity | Conc. | Viscosity | Conc. | Viscosity | Conc. | Viscosity |
| (mg/mL) | (cP) | (mg/mL) | (cP) | (mg/mL) | (cP) | (mg/mL) | (cP) |
| 89.1 | 3.4 | 90.5 | 2.8 | 92.1 | 2.8 | 93.2 | 2.8 |
| 114.3 | 5.3 | 105.9 | 3.4 | 113.7 | 3.6 | 118.6 | 3.8 |
| 126.2 | 6.7 | 115.3 | 4.2 | 115.3 | 3.8 | 123.4 | 4.2 |
| 128.7 | 6.8 | 131.5 | 5.1 | 133.1 | 5.0 | 135.8 | 5.3 |
| 135.6 | 8.5 | 131.5 | 5.5 | 134.1 | 5.1 | 138.0 | 5.5 |
| 139.9 | 9.0 | 143.9 | 7.1 | 143.6 | 6.3 | 150.4 | 7.4 |
| 157.3 | 10.8 | 152.7 | 7.7 | 143.9 | 6.2 | 154.7 | 7.0 |
| 158.3 | 11.6 | 155.5 | 8.9 | 156.6 | 8.2 | 163.7 | 8.4 |
| 162.2 | 14.2 | 159.5 | 9.0 | 158.0 | 7.4 | 167.3 | 7.9 |
| 167.4 | 16.1 | 167.0 | 11.0 | 173.4 | 10.6 | 181.3 | 11.2 |
| 173.9 | 19.0 | 174.7 | 12.8 | 174.1 | 10.5 | 182.0 | 12.0 |
| 174.1 | 17.4 | 174.9 | 11.7 | 179.3 | 11.8 | 189.3 | 12.8 |
| 181.3 | 21.3 | 179.9 | 14.7 | 183.6 | 12.3 | 190.2 | 13.3 |
| 183.1 | 22.1 | 183.9 | 15.0 | 187.8 | 13.3 | 192.3 | 14.5 |
| 194.6 | 28.7 | 193.0 | 17.1 | 189.5 | 13.8 | 208.0 | 16.5 |
| 197.2 | 29.9 | 196.0 | 19.3 | 202.2 | 18.6 | 213.4 | 19.3 |
| 206.9 | 37.2 | 206.8 | 22.5 | 205.3 | 19.5 | | |
| 216.9 | 48.1 | | | | | | |

Injection force data presented in table below shows that injection force depends on formulation, protein concentration, inner needle diameter and injection time. All experiments were performed at room temperature around 18-20° C. In this study most combinations of the tested formulations, needle thickness and injection time revealed an injection force <25 Newton. 1 mL of anti IL22R at 200 mg/mL in F11_s3 and F11_s4 can easily be injected through a 27G STW needle with a preset duration of 5 sec. The force ˜18 Newton (for 1 mL during 5 seconds) corresponds to the force needed to inject 2 mL (400 mg) within 10 seconds.

| | Injection force | | | |
|---|---|---|---|---|
| | (newton) | | | avr. |
| F11_r 150 mg/ml, 27G needle, 1 mL injection | 12.3 | 11.2 | 11.7 | 11.7 |
| in 5 sec. (n = 3) | | | | |
| F11_r 175 mg/ml, 27G needle, 1 mL injection | 19.9 | 14.9 | 17.4 | 17.4 |
| in 5 sec. (n = 3) | | | | |
| F11_s2 175 mg/ml, 27G needle, 1 mL injection | 13.2 | 10.1 | 11.6 | 11.6 |
| in 5 sec. (n = 3) | | | | |
| F11_s3 175 mg/ml, 27G needle, 1 mL injection | 11.8 | 11.5 | 11.6 | 11.6 |
| in 5 sec. (n = 3) | | | | |
| F11_s4 175 mg/ml, 27G needle, 1 mL injection | 11.6 | 9.0 | 10.3 | 10.3 |
| in 5 sec. (n = 3) | | | | |
| F11_s3 200 mg/ml, 27G needle, 1 mL injection | 17.9 | 13.9 | 15.9 | 15.9 |
| in 5 sec. (n = 3) | | | | |
| F11_s4 200 mg/ml, 27G needle, 1 mL injection | 18.6 | 14.3 | 16.4 | 16.4 |
| in 5 sec. (n = 3) | | | | |
| F11_r 150 mg/ml, 29G needle, 1 mL injection | 24.8 | | | |
| in 5 sec. (n = 1) | | | | |
| F11_s3 175 mg/mL, 29G needle, 1 mL | 25.4 | | | |
| injection in 5 sec. (n = 1) | | | | |
| F11_s4 175 mg/mL, 29G needle, 1 mL | 23.6 | | | |
| injection in 5 sec. (n = 1) | | | | |
| F11_s3 200 mg/mL, 29G needle, 1 mL | 39.1 | | | |
| injection in 5 sec. (n = 1) | | | | |
| F11_s2 175 mg/ml, 27G needle, 1 mL injection | 6.5 | | | |
| in 10 sec. (n = 1) | | | | |
| F11_s3 175 mg/mL, 27G needle, 1 mL | 6.3 | | | |
| injection in 10 sec. (n = 1) | | | | |

### Example 20: Stability at 30° C. and 40° C.

Anti IL22R formulations prepared for the experiment outlined in example 19 were also used to evaluate anti IL22R stability at 30° C. and 40° C. (data presented in the present example). It has been shown that anti IL22R stability at elevated temperatures ≥40° C. is compromised by addition of arginine (ref. example 6, 10, 12, 13), however less clear effect of arginine on stability was observed at lower temperatures as shown and discussed in example 18. Samples used for this experiment were prepared according to the procedure described in example 19.

40° C. stability. The SEC data shows that at 40° C. the most stable formulation (in terms of having lowest amount of protein aggregates, lowest HMWP %) is F11_r at anti IL22R concentration 151.2 mg/mL. It is most relevant to compare formulations at similar concentrations. Comparison of formulations containing ˜175 mg/mL anti IL22R shows that anti IL22R after 4 weeks storage at 40° C. is most stable in F11_r, followed by F11_s2 and F11_s3, followed by F11_s4 as the least stable at 40° C. Comparison of F11_s3 and F11_s4 at ˜200 mg/mL shows that anti IL22R is most stable in F11_s3.

40° C. stability. The stability of anti IL22R at 40° C. seems to depend on the concentration of arginine and trehalose. The stability is highest at lowest arginine concentration (highest trehalose concentration). Hence F11_r is a formulation providing the most thermostable anti IL22R antibody. The viscosity of anti IL22R in F11_r >30 cp at room temperature when the protein concentration is 200 mg/ml (ref. example 14 and 19).

30° C. stability. The SEC data shows that at 30° C. there are only very small differences between formulations in terms of anti IL22R stability. At 175 mg/mL anti IL22R there is no correlation between stability and arginine concentration (as observed at 40° C.), at 200 mg/ml F11_s3 seems to be slightly more stable compared to F11_s4 (which is in line with long term data at 25° C., example 18).

| HMWP % | T0 | 1 w 30° C. | 2 w 30° C. | 4 w 30° C. | 1 w 40° C. | 2 w 40° C. | 4 w 40° C. |
|---|---|---|---|---|---|---|---|
| F11_r 151.2 | 1.5 | 1.6 | 1.7 | 2.0 | 2.3 | 2.8 | 3.5 |
| mg/mL | | | | | | | |
| F11_r 174.1 | 1.5 | 1.7 | 1.9 | 2.2 | 2.6 | 3.2 | 4.2 |
| mg/mL | | | | | | | |
| F11_s2 175.6 | 1.3 | 1.4 | 1.6 | 1.9 | 3.1 | 3.6 | 4.9 |
| mg/mL | | | | | | | |
| F11_s3 174.9 | 1.3 | 1.5 | 1.6 | 2.0 | 3.2 | 3.7 | 4.9 |
| mg/mL | | | | | | | |
| F11_s4 174.7 | 1.3 | 1.5 | 1.7 | 2.0 | 3.3 | 4.0 | 5.7 |
| mg/mL | | | | | | | |
| F11_s3 202.4 | 1.4 | 1.6 | 1.7 | 2.2 | 3.7 | 4.4 | 5.9 |
| mg/mL | | | | | | | |
| F11_s4 201.4 | 1.3 | 1.6 | 1.8 | 2.3 | 3.8 | 4.7 | 6.9 |
| mg/mL | | | | | | | |

All anti IL22R samples/formulations used to generate data for the present examples were prepared with milliQ water or water for injection (WFI). All pH measurement were conducted at room temperature with calibrated equipment.

### Example 21: Effects of Tween 20 (Polysorbate 20) on Sub Visible Particle (SVP) Formation in Aged Anti IL22R Formulations in Prefilled Syringes

Polysorbate 20 was tested for its effects on sub-visible particle formation in anti-IL22R formulations at high protein concentration. The analyses were conducted on aged formulations as well as on aged and agitated formulations to increase the air-liquid interphasic stress. The effect of polysorbate 20 was evaluated by microfluidic imaging (MFI, FlowCam). Anti-IL22R at 150±15 mg/ml in 20 mM histidine, 80 mM glycine, 20 mM methionine, 180 mM trehalose, pH 6.0 was manufactured. After manufacturing polysorbate 20 was spiked into part of the material resulting in two different formulations: F1 with 0.02% PS20 (2 mg/ml polysorbate 20) and F2 without surfactant. The formulations were filled into PFS' and hereafter exposed to long term storage at 5° C. The agitation study was performed after 3 years storage at 5° C. Agitation was performed using a 2D shaking board at 200 RPM for 3 days (˜72 hours) at room temperature (19-23° C.), syringes were agitated both laying on the side and in needle-up position. Not agitated (quiescent) control was included in the same room.

Analyses: The analysis was performed using FlowCam 8100 equipped with automated liquid handler and 10× magnication objective. Formulatioins were transferred into a 96-well plate and analysed undiluted. For each measure 150 μl (corresponding to approximately 75 μl analytical volume) was used. Measures invalidated by background calibration issues or critical fluidity issues where discarded. Particles with circularity ≥0.85 (e.g. air bubbles) were filtered out during data analysis.

Results. The table below shows SVP counts pr mL (mean+/−standard deviation) divided into different SVP size groups. N refers to the number of independent measures.

| | | SVP /ml | | | |
|---|---|---|---|---|---|
| Formulation | Treatment | >5 μm | >10 μm | >25 μm | N |
| F1 (PS20 0.02%) | Control (no agitation) | 2031 ± 183 | 194 ± 61 | 49 ± 40 | 7 |
| F2 (No-surf.) | Control (no agitation) | 17619 ± 3336 | 6348 ± 1487 | 744 ± 383 | 4 |
| F1 (PS20 0.02%) | Lay on side, 72 hrs agitation, | 2406 ± 1111 | 236 ± 113 | 38 ± 30 | 9 |
| | 200 RPM | | | | |
| F2 (No-surf.) | Lay on side, 72 hrs agitation, | 19834 ± 6947 | 6062 ± 2614 | 657 ± 527 | 6 |
| | 200 RPM | | | | |
| F1 (PS20 0.02%) | Needle up, 72 hrs agitation, | 2680 ± 657 | 237 ± 80 | 21 ± 27 | 6 |
| | 200 RPM | | | | |
| F2 (No-surf.) | Needle up, 72 hrs agitation, | 15679 ± 4419 | 5178 ± 1333 | 425 = 119 | 2 |
| | 200 RPM | | | | |
| Placebo F1 | Control (no agitation) | 3474 ± 169 | 218 ± 39 | 18 ± 13 | 10 |
| (background) | | | | | |
| Placebo F1 | Lay on side, 72hrs agitation, | 2608 ± 717 | 87 ± 47 | 6 ± 10 | 9 |
| (background) | 200 RPM | | | | |
| Placebo F2 | Lay on side, 72hrs agitation, | 125 ± 75 | 49 ± 37 | 9 ± 13 | 11 |
| (background) | 200 RPM | | | | |

Extensive amount of SVP was observed in the F2 formulation (without surfactant) compared to the F1 formulation with 0.02% PS20. Roughly about ˜5-30 times as many SVP's was observed in F2 compared to F1 (the biggest difference in particle counts was observed for the larger particles). No clear difference in SVP counts was observed between the protein free F1 and F2 formulations (placebo F1 and placebo F2). Morphological examination of FlowCam SVP images revealed that none or very few particles in F1 seemed proteinaceous. Conversely, the morphology of many SVPs in F2 suggested proteinaceous type particles. The difference between F1 and F2 observed by FlowCam seems to be mostly independent on agitation. In other words, particle counts in F1 and F2 respectively are very similar before and after agitation. The effect of polysorbate 20 SVP formation in PFS was clear already before agitation (control samples).

### Example 22: Effects of Surfactants on Sub Visible Particle (SVP) Formation in Anti IL22R Formulations in 2R Vials

Non-ionic surfactants (Polysorbate 20, Polysorbate 80, Poloxamer 188) were tested for their effects on sub-visible particle formation in anti-IL22R formulations at high protein concentration. The influence of surfactants in the range of 0.01-0.04% (w/w) (same as 1˜4 mg/ml) was evaluated by orthogonal techniques (size exclusion chromatography, dynamic light scattering, light obscuration, and microfluidic imaging), this example presents the microfluidic imaging (MFI) data. Anti IL22R at 150±15 mg/ml in 20 mM histidine, 80 mM glycine, 20 mM methionine, 180 mM trehalose, pH 6.0 was sampled during up-scaled manufacturing. Hereafter surfactant stock solutions were spiked into the formulation to obtain various samples with different surfactants at different concentrations (0.01-0.04% (w/w). 1.6 mL of each formulation was filled into 2R vials. The experimental analyses were conducted on freshly prepared formulations (not on stability samples as in previous example) as well as on formulations exposed to agitation to increase the air-liquid interphasic stress. Agitation was started (placing the 2R vials on an agitation plate) within 24 hours after surfactant spike. Agitation was performed using a 2D shaking board at 200 RPM for 3 days (˜72 hours) at room temperature (19-23° C.). Not agitated control was placed in the same room.

Analyses: Data presented in this example is based on analysis performed using FlowCam 8100 equipped with automated liquid handler and 10× magnication objective. Formulatioins were transferred into a 96-well plate and analysed undiluted. For each measure 150 μl (corresponding to approximately 75 μl analytical volume) was used. Measures invalidated by background calibration issues or critical fluidity issues were discarded. Particles with circularity ≥0.85 (e.g. air bubbles) where filtered out during data analysis.

Results. The table below shows SVP counts pr mL (mean+/−standard deviation) divided into different SVP size groups. N refers to the number of independent measures.

| Formulation | Treatment | SVP ≥ 5 μm | SVP ≥ 10 μm | SVP ≥ 25 μm | N |
|---|---|---|---|---|---|
| Placebo | Control | 1493 ± 1318 | 588 ± 578 | 113 ± 124 | 138 |
| (backgorund) | | | | | |
| No surfactant | Control (no agitation) | 3723 ± 1755 | 1284 ± 794 | 235 ± 211 | 8 |
| No surfactant | 24 hrs agitation at 200 | 6064 ± 2154 | 2366 ± 955 | 335 ± 178 | 7 |
| | RPM | | | | |
| No surfactant | 48 hrs agitation at 200 | 10874 ± 4549 | 5527 ± 3377 | 2651 ± 2868 | 6 |
| | RPM | | | | |
| No surfactant | 72 hrs agitation at 200 | 34509 ± 18198 | 17716 ± 9455 | 5531 ± 5929 | 8 |
| | RPM | | | | |
| PS80 0.01% (w/w) | Control (no agitation) | 450 ± 251 | 154 ± 82 | 19 ± 18 | 9 |
| PS80 0.01% (w/w) | 72 hrs agitation at 200 | 836 = 326 | 204 ± 62 | 27 ± 20 | 10 |
| | RPM | | | | |
| PS20 0.02% (w/w) | Control (no agitation) | 528 ± 268 | 138 ± 74 | 27 ± 29 | 16 |
| PS20 0.02%(w/w) | 72 hrs agitation at 200 | 653 ± 476 | 138 ± 122 | 19 ± 23 | 15 |
| | RPM | | | | |
| P188 0.01%(w/w) | Control (no agitation) | 452 ± 305 | 104 ± 56 | 30 ± 30 | 8 |
| P188 0.01%(w/w) | 72 hrs agitation at 200 | 4669 ± 451 | 1844 ± 296 | 281 ± 107 | 8 |
| | RPM | | | | |
| P188 0.02%(w/w) | Control (no agitation) | 529 ± 574 | 147 ± 164 | 15 ± 18 | 8 |
| P188 0.02%(w/w) | 72 hrs agitation at 200 | 439 ± 133 | 159 ± 58 | 38 ± 40 | 8 |
| | RPM | | | | |
| P188 0.04%(w/w) | Control (no agitation) | 310 ± 143 | 105 ± 55 | 15 ± 20 | 8 |
| P188 0.04%(w/w) | 72 hrs agitation at 200 | 732 ± 317 | 289 ± 156 | 49 ± 40 | 8 |
| | RPM | | | | |

Comparison of FlowCam data on control samples (not agitated samples) show that presence of surfactants in anti IL22R formulations significantly reduces the number of sub visible particles. The number of particles in freshly prepared samples (no agitated) is roughly about 10-20 times higher for samples without surfactants. However, the effects of surfactants are even more pronounced for agitated samples. The general observation is that the effect of surfactants is very significant in terms of reducing SVP, especially the formation of lager particles during agitation is reduced by surfactants. For particles >25 μm addition of 0.02% (w/w) polysorbate 20 (Tween 20) reduces the number about 300 times. Almost the same effect is observed for polysorbate 80 at 0.01% (w/w) and poloxamer 188 at 0.02-0.04% (w/w). Poloxamer 188 reduced SVP formation in the tested range 0.01%-0.04% (w/w) and was found most effective in terms of preventing SVP formation at 0.02% (w/w).

In summary, the presence of surfactants, that is polysorbate 20, polysorbate 80 and poloxamer 188 is demonstrated to stabilize anti IL22R formulations in a way that inhibit SVP formation.

1. A liquid pharmaceutical formulation, comprising an IL-22R antibody at a concentration 150±15 mg/mL-200 mg/mL±25 mg/mL, and further comprising:
one or more disaccharides,
one or more amino acid,
a buffer,
an antioxidant,
optionally a viscosity lowering agent,
optionally a surfactant,
at pH 5.5-6.5, having an osmolality of 280-450 mOsm/kg.

2. The pharmaceutical formulation of claim 1, comprising an IL-22R antibody at a concentration 150±15 mg/mL-200 mg/mL±25 mg/mL, and further comprising:
one or more disaccharides at total concentrations of 60-260 mM,
one or more amino acid, selected from the group consisting of glycine, proline, lysine,
glutamic acid, methionine, arginine, aspartic acid, and histidine.
at total concentration of 40-140 mM,
optionally a surfactant,
at pH 5.5-6.5, having an osmolality of 280-450 mOsm/kg.

3. The pharmaceutical formulation of any one of claims 1-2, comprising an IL-22R antibody at a concentration 150±15 mg/mL-200 mg/mL±25 mg/mL, and further comprising:
one or more disaccharides at total concentrations of 60-260 mM,
one or more amino acids selected from the group consisting of glycine, proline, lysine,
glutamic acid, methionine, arginine, aspartic acid, and histidine.
at total concentration of 40-140 mM, wherein the amino acids functions as stabilizer,
antioxidant, viscosity lowering agent and buffer,
optionally a surfactant,
at pH 5.5-6.5, having an osmolality of 280-450 mOsm/kg.

4. The pharmaceutical formulation according to any of the claims 1-3, comprising an IL-22R antibody at a concentration 150±15 mg/mL-200 mg/mL±25 mg/mL, and further comprising:
one or more disaccharides at total concentrations of 60-260 mM,
one or more amino acids selected from the group consisting of glycine, proline, lysine,
glutamic acid, methionine, arginine, aspartic acid, and histidine.
at total concentration of 40-140 mM, wherein
proline and/or glycine are stabilizers,
methionine is an anti-oxidant,
arginine is a viscosity lowering agent,
histidine is a buffer, and
optionally a surfactant,
at pH 5.5-6.5, having an osmolality of 280-450 mOsm/kg.

5. The pharmaceutical formulation according to any one of claims 1-4, wherein a surfactant is present at a concentration of 0.01-0.04% (w/w),

6. The pharmaceutical formulation of claim 5, wherein the surfactant is a non-ionic surfactant.

7. The pharmaceutical formulation of claim 6, wherein the surfactant is selected from the group consisting of Polysorbate 20, Polysorbate 80 and Poloxamer 188.

8. The pharmaceutical formulation according to claim 1, comprising an IL-22R antibody at a concentration 150±15 mg/mL-200 mg/ml±25 mg/mL, and further comprising:
one or more disaccharides at total concentrations of 60-260 mM,
proline and/or glycine are present at a concentration of 0-80 mM,
methionine is present in a concentration of 5-30 mM,
arginine is present in a concentration of 0-100 mM,
histidine is present in a concentration of 0-30 mM, and
optionally a surfactant,
at pH 5.5-6.5, having an osmolality of 280-450 mOsm/kg.

9. The pharmaceutical formulation according to claim 8, comprising an IL-22R antibody at a concentration 150±15 mg/mL-200 mg/mL±25 mg/mL, and further comprising:
one or more disaccharides at total concentrations of 60-260 mM,
glycine is present at a concentration of 0-80 mM,
methionine is present in a concentration of 5-30 mM,
arginine is present in a concentration of 0-100 mM,
histidine is present in a concentration of 0-30 mM, and
optionally a surfactant,
at pH 5.5-6.5, having an osmolality of 280-450 mOsm/kg.

10. The pharmaceutical formulation according to any one of claims 8-9 wherein a surfactant is present at a concentration of 0.01-0.04% (w/w),

11. The pharmaceutical formulation according to claim 10, wherein a surfactant is present at a concentration of 0.01-0.04% (w/w),

12. The pharmaceutical formulation according to claim 10, wherein the surfactant is selected from the group consisting of polysorbate 20, polysorbate 80 and poloxamer 188.

13. The pharmaceutical formulation according to any of the claims 1-12 wherein the formulation contains a histidine buffer.

14. The pharmaceutical formulation according claim 13 wherein histidine is present in a concentration of 20 mM.

15. The pharmaceutical formulation according to any of the claims 1-14, wherein the disaccharide is trehalose or sucrose.

16. The pharmaceutical formulation according to claim 15 wherein the disaccaride is trehalose.

17. The pharmaceutical formulation according to any of the claims 1-116, wherein the surfactant is present in a concentration of 0.01-0.02% (w/w),

18. The pharmaceutical formulation according to any of the claims 1-17, wherein the surfactant is polysorbate 20.

19. The pharmaceutical formulation according any of the claims 1-18 wherein the anti-oxidant methionine is present in a concentration of 20 mM.

20. The pharmaceutical formulation of claim 1, comprising an IL-22R antibody at a concentration 150 mg/ml±15 mg/mL, and
one or more disaccharides at total concentrations of 180-260 mM,
one or more amino acids at total concentration of 40-120 mM,
an anti-oxidant at a concentration of 5-30 mM,
a viscosity lowering agent at a concentration of 0-100 mM,
optionally a surfactant at a total concentration of 0.01-0.04% (w/w),
a histidine buffer at a concentration of 20 mM,
at pH 5.6-6.5, having a osmolality of 280-450 mOsm/kg

21. The pharmaceutical formulation of claim 21, wherein a surfactant is present at a concentration of 0.01-0.03% (w/w).

22. The pharmaceutical formulation according to claim 21, comprising an IL-22R antibody at a concentration 150 mg/mL±15 mg/mL, and
trehalose at total concentrations of 180-260 mM,
glycine at total concentration of 0-80 mM,
methionine at a concentration of 5-30 mM,
polysorbate 20 at a total concentration of 0.01-0.03% (w/w),
a histidine buffer at a concentration of 20 mM,
at pH 5.6-6.5, having a osmolality of 280-450 mOsm/kg

23. The pharmaceutical formulation according to claim 21, comprising an IL-22R antibody at a concentration 150 mg/mL±15 mg/mL, and
trehalose at total concentrations of 180-260 mM,
glycine at total concentration of 0-80 mM,
methionine at a concentration of 5-30 mM,
polysorbate 80 at a total concentration of 0.01-0.03% (w/w),
a histidine buffer at a concentration of 20 mM,
at pH 5.6-6.5, having a osmolality of 280-450 mOsm/kg

24. The pharmaceutical formulation according to claim 21, comprising an IL-22R antibody at a concentration 150 mg/mL±15 mg/mL, and
trehalose at total concentrations of 180-260 mM,
glycine at total concentration of 0-80 mM,
methionine at a concentration of 5-30 mM,
poloxamer 188 at a total concentration of 0.01-0.03% (w/w),
a histidine buffer at a concentration of 20 mM,
at pH 5.6-6.5, having a osmolality of 280-450 mOsm/kg.

25. The pharmaceutical formulation according to any of the claims 21-24, comprising an IL-22R antibody at a concentration 150 mg/ml±15 mg/mL, and
trehalose at total concentrations of about 180 mM,
glycine at total concentration of about 80 mM,
methionine at a concentration of about 20 mM,
polysorbate 20 20 at a total concentration of about 0.01-0.03% (w/w),
a histidine buffer at a concentration of about 20 mM,
at pH 5.6-6.5.

26. A liquid pharmaceutical formulation according to claim 1, comprising an IL-22R antibody at a at a concentration 200 mg/mL±25 mg/mL, and further comprising:
trehalose at total concentrations of 60-100 mM,
methionine at a concentration of 5-30 mM,
viscosity lowering agent at a concentration of 60-100 mM,
glycine at concentration of 0-80 mM
optionally a surfactant
at pH 5.6-6.5, having a tonicity of 280-450 mOsm/kg.

27. The pharmaceutical formulation according to claim 26, comprising an IL-22R antibody at a concentration of 200 mg/mL±25 mg/mL, and further comprising:
trehalose at total concentrations of 80-100 mM,
methionine at a concentration of 5-30 mM,
arginine at a concentration of 60-100 mM,
a surfactant at a total concentration of 0.01-0.03% (w/w),
a histidine buffer at a concentration of 20 mM, at pH 5.5-6.5, having an tonicity of 280-450 mOsm/kg

28. The pharmaceutical formulation according to claim 26, comprising an IL-22R antibody at a concentration of 200 mg/mL±25 mg/mL, and further comprising:
trehalose at total concentrations of 80-100 mM,
methionine at a concentration of 5-30 mM,
arginine at a concentration of 60-100 mM,
0.00% surfactant,
a histidine buffer at a concentration of 20 mM, at pH 5.5-6.5, having an tonicity of 280-450 mOsm/kg

29. The pharmaceutical formulation according to claims 26-27, comprising an IL-22R antibody at a concentration 200 mg/ml±25 mg/mL, and further comprising:
trehalose at total concentrations of about 100 mM,
methionine at a concentration of about 20 mM,
arginine at a concentration of about 80 mM,
polysorbate 20 at a total concentration of about 0.02% (w/w),
histidine buffer at a concentration of about 20 mM,
at pH 5.5-6.5.

30. The pharmaceutical formulation according to claims 26-27, comprising an IL-22R antibody at a concentration 200 mg/mL±25 mg/mL, and further comprising:
trehalose at total concentrations of about 100 mM,
methionine at a concentration of about 20 mM,
arginine at a concentration of about 80 mM,
polysorbate 80 at a total concentration of about 0.02% (w/w),
histidine buffer at a concentration of about 20 mM,
at pH 5.5-6.5.

31. The pharmaceutical formulation according to claims 26-27, comprising an IL-22R antibody at a concentration 200 mg/mL±25 mg/mL, and further comprising:
trehalose at total concentrations of about 100 mM,
methionine at a concentration of about 20 mM,
arginine at a concentration of about 80 mM,
poloxamer 188 at a total concentration of about 0.02% (w/w),
histidine buffer at a concentration of about 20 mM,
at pH 5.5-6.5.

32. The pharmaceutical formulation according to any of the claims 1-31 which is stable at 5° C. for 3 years.

33. The pharmaceutical formulation according to any of the claims 1-31 which is stable at 5° C. for 2 years.

34. The pharmaceutical formulation according to any of the claims 1-31 for use in the treatment of atopic dermatitis.

35. The pharmaceutical formulation according to any of the claims 1-31 wherein the IL-22R antibody is defined by the CDR sequences HCDR1: SEQ ID No 1, HCDR2: SEQ ID No 2, HCDR3: SEQ ID No 3, LCDR1: SEQ ID No 4, LCDR2: SEQ ID No 5 and LCDR3: SEQ ID No 6.

36. A method of treating atopic dermatitis in a subject in need thereof, comprising administration of an IL-22R antibody in a formulation according to any of the claims 1-31.


SEQUENCE LISTING (10 sequences)

SEQ ID NO: 1 - protein, 5 aa, synthetic construct
SYDMN

SEQ ID NO: 2 - protein, 17 aa, synthetic construct
SIYNDASNTAYSDSVKG

SEQ ID NO: 3 - protein, 11 aa, synthetic construct
VGFSGTYYSES

SEQ ID NO: 4 - protein, 7 aa, synthetic construct
QGGYYAH

SEQ ID NO: 5 - protein, 7 aa, synthetic construct
GQNNRPS

SEQ ID NO: 6 - protein, 10 aa, synthetic construct
QSGSSSSNAV

SEQ ID NO: 7 - protein, 450 aa, synthetic construct
QVQLVESGGGLVQPGGSLRLSCAASGFTFSSYDMNWVRQAPGKGLEWVSSIYNDASNTAYSDSVKGRFTISRDNSKNTLYLQMNSLRAEDTAVYYCAKVGFSGTYYSESWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 209 aa, synthetic construct
SYELTQPSSVSVALGQTARITCQGGYYAHWYQQKPGQAPVLVIYGQNNRPSGIPERFSGSGAGNTATLTISRAQAEDEADYYCQSGSSSSNAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 120 aa, synthetic construct
QVQLVESGGGLVQPGGSLRLSCAASGFTFSSYDMNWVRQAPGKGLEWVSSIYNDASNTAYSDSVKGRFTISRDNSKNTLYLQMNSLRAEDTAVYYCAKVGFSGTYYSESWGQGTLVTVSS

SEQ ID NO: 10 - protein, 103 aa, synthetic construct
SYELTQPSSVSVALGQTARITCQGGYYAHWYQQKPGQAPVLVIYGQNNRPSGIPERFSGSGAGNTATLTISRAQAEDEADYYCQSGSSSSNAVFGGGTKLTVL